(19) United States
(12) Patent Application Publication
(10) Pub. No.: US 2008/0226565 A1
(43) Pub. Date: Sep. 18, 2008

(54) Anticariogenic Proteins & Peptides & Saccharides

(76) Inventor: Lucas Huybrechts, Kontich (BE)

Correspondence Address: CLARK & ELBING LLP, 101 FEDERAL STREET, BOSTON, MA, 02110, US

(21) Appl. No.: 10/586,563

(22) PCT Filed: Nov. 29, 2004
(86) PCT No.: PCT/EP2004/013549

§ 371 Date: Feb. 29, 2008

(30) Foreign Application Priority Data
Jan. 19, 2004 (BE) 2004/0028

(51) Int. Cl. A61K 8/44; A61K 31/198; A61Q 11/00

(52) U.S. Cl. 424/49; 514/563

(57) ABSTRACT

This Invention discloses new proteins, peptides and saccharides that have anticarogenic capabilities and that are characterized by the presence of one or more components that have the ability to form a complex with calcium ions: such as epsilon-polylysine that is conjugated with one or more bisphosphonyl-, biscarboxyl-, or 3-hydroxy-phthalate-groups or conjugated with casein phosphopeptide, phosvitin or with partially hydrolyzed phosvitin; such as partially hydrolized chitosan that is conjugated with one or more bisphosphonyl groups, casein phosphopeptide or with phosvitin or partially hydrolized phosvitin; such as bisphosphonylated and biscarboxylated proteins with at least 40% of amino acids consisting of lysine and a molecular weight of above 2 kD and such as polymerized casein phosphopeptide and partially hydrolized phosvitin. The products can be used in formulations to protect teeth and to treat the oral cavity. The Invention encompasses competent protein, peptide and saccharide structures, as well as production procedures and application conditions.

### FIELD OF INVENTION

Caries is caused by oral bacteria that produce acid which is dissolving the surface of the tooth.

The present invention relates to proteins, peptides and saccharides that have the ability to protect teeth against acid attack. The invention discloses the appropriate molecular structures that are required to generate protection capability, the production processes for said protecting agents and the development of end-formulations for use in the protection of teeth.

### BACKGROUND OF THE INVENTION

It is well known that caries is caused by acid which is produced by oral bacteria. Sugars, such as glucose, are an important food source for the bacteria and their availability is required for the production of acid. Streptococcus mutans (S. mutans) is considered as the most important culprit in the occurrence of caries, survives well in the oral cavity and produces relative large amounts of acid. S. mutans also produces glucosyltransferase enzymes that catalyse the production of sticky glucanes and fructanes, which are used by the bacteria for improving attachment to teeth and plaque. The saliva effectively can protect teeth partially; it contains antibacterial agents, buffering ingredients and calcium and phosphate building blocks that can be used for repair work. Notwithstanding, the protection competence of saliva is not sufficient and the tooth surface will be demolished and gradually demineralized as a result of regular acid production. Over time, holes will appear that may finally result in the loss of the teeth.

The use of fluoride for the reduction of caries has been grown enormously during the last forty years. Today flouride containing toothpastes are available worldwide. Many scientific studies have demonstrated that fluoride has the ability to (at least partially) inhibit caries; a competence based on a reduction of the demineralization process and re-enforcement of the remineralization process (U.S. Pat. No. 5,089,255; Dental Caries, O. Fejerskow et al. Blackwell Munksgaard, 2003, ISBN 1-4051-0718-9). The effect of fluoride can be enhanced with the use of co-ingredients such as zinc-components (U.S. Pat. No. 4,396,599). Notwithstanding, the protecting & inhibiting effect of fluoride is limited to approx. 25% of caries that would occur if no use would be made of fluoride. Even today the number of people in the world that are affected with caries is enormous. In addition, the toxicological profile of fluoride is under debate (especially for it's carcinogenic, inflammation, lung damaging, immunological, fluorosis and bone deformation potential). Some European countries have banned fluoride supplements from the market and the U.S. Food and Drug Administration (FDA) has limited it's use in toothpaste for home-use at 1150 ppm maximum.

More recently, several strategies have been explored in the search to new protecting agents: 1. Destruction of bacteria, 2. Reduction of the production of acid, 3. Prevention of attachment of bacteria to the tooth surface and the plaque, 4. Repair of the tooth surface with calcium and phosphate building blocks and 5. the absorption of acid at the tooth surface.

1. Destruction of bacteria. Many agents with antibacterial activity against bacteria from the oral cavity, have been discovered; this includes lantibiotic peptides from bacteria (U.S. Pat. No. 4,209,508; US pending 20030118590), lytic enzymes from bacteriophages that are able to destroy the cell wall of streptococcus mutans (U.S. Pat. No. 6,355,012, U.S. Pat. No. 6,399,098), extracts from yeast-cells (U.S. Pat. No. 4,980,151) or components from natural oils such as Cubeb oil or java citronella oil (U.S. Pat. No. 4,590,215: alpha cadinol). Also extracts from plants as Angelica Gigas, Akebia quinata, Camellia sinensis and Korean Ginseng Flower. (J. Dent. Res. 2003; vol 82, spec. issue B, 1591 and 1670) appear to demonstrate antibiotic activity. In addition synthetic antibacterial agents have been developed that act against streptococcus mutans: N-alkyl-pyridinum chloride, bisbiguanides (Chlorohexidine, U.S. Pat. No. 6,143,281), quaternary ammonium-galactomannans (U.S. Pat. No. 4,282,204), polyphenols (triclosan; U.S. Pat. No. 6,136,298) and modified histatine-peptides (U.S. Pat. No. 5,672,351). Other strategies consist in the stimulation of an immunological reaction against S. Mutans with peptides that have an identical structure compared to parts of the S. Mutans antigen I/II (cell wall protein; U.S. Pat. No. 6,024,958 and U.S. Pat. No. 6,500,433), or the use of antibodies against S. Mutans antigen I/II that have been produced with another bacteria (U.S. Pat. No. 5,612,031). Some suggest to use bacteriophages in the fight against S.Mutans (U.S. Pat. No. 4,957,686 and U.S. Pat. No. 4,891,210).
2. The production of acid can be limited with the use of sugars such as erythritol (U.S. Pat. No. 6,238,648, U.S. Pat. No.6,177,064, U.S. Pat. No. 4,346,116), xylitol, trehalose, palatinose (U.S. Pat. No. 5,985,622). Despite their large-scale use in food, it is necessary to replace large amounts of glucose, and the use of an elevated dosage may result in an unwanted laxative side effect.
3. The attachment of S. Mutans to the tooth surface could possibly be limited in a variety of ways: Pyrophosphates and ZnO limit the formation of plaque and reduces the attachment possibilities (U.S. Pat. No. 5,455,024); glucanases and dextranases degrade glucanes that are used by the bacteria to attach to plaque (U.S. Pat. No. 5,468,479); it could be possible to use a peptide vaccin that stimulates an immunological reaction against glucosyltransferase; this enzym catalyses the production of glucanes (U.S. Pat. No. 5,686,075); peptides with a structure similar to parts of the S. Mutans antigen I/II could compete with the antigen for attachment to plaque (U.S. Pat. No. 6,500,433).
4. Repair work. Saliva contains calcium and phosphate ions that contribute to the repair of the tooth surface. It does also contain natural proteins (e.g. statherin) that helps preventing the precipitation of such ions, keeping them available for repair work. Casein (U.S. Pat. No. 5,130,123) and especially also non-denaturated casein (U.S. Pat. No. 5,427,769) have anticariogenic competence.

Caseinphoshopeptides (CPP) are made by hydrolyses of casein, and they too have anticariogenic competence. CPP forms a complex with calcium and phosphate ions, and stimulates repair work.

5. Adhesion to the tooth surface & acid absorption.

Adhesion allows the product to accumulate at the location where it is needed, the tooth surface. Caseinphosphopeptide as well as phosvitin, a protein found in egg, have the ability to attach; they accumulate on the surface and generate a local buffering capability that contributes to preventing the acid to dissolve the tooth (U.S. Pat. No. 5,279,814 en B. Jiang et al, Biosci. Biotechnol. Biochem., 65 (5), 1187-1190,2001 en B. Jiang et al, J. Agric. Food Chem.,48, 990-994,2000 en A. Goulas et al, J. of Protein Chemistry, vol 15, no. 1, 1-9,1996).

Products with basic components have buffering competence. Arginine and small peptides (2-4) that contain at least one arginine amino acid, have been used to increase the pH of the plaque (U.S. Pat. No. 621851, U.S. Pat. No. 4,225,579, U.S. Pat. No. 5,762,911 en Kanapka, Archs. oral. Biol. 28, 1007-1015, 1983). Also chitosan (salts) are recommended (U.S. Pat. No. 4,512,968); but their potential is restrained by lack of solubility at neutral or basic pH. Another polyamine, polyethyleneimino fluorophosphate appears to attach to teeth and to protect against acid (U.S. Pat. No. 4,020,019). However, it's resistance against enzymatic degradation and it's capability to attach to a cell wall, renders it irritant and toxic.

The occurrence of caries is still widespread, despite the many creative research efforts that have been carried out; 99% of Germans and 94% of Italians between 35-44 years of age and 40% of Belgian children between 5-7 years of age, suffer from caries according to an epidemiological study that has been carried out in Europe a few years ago. It is therefore necessary that new products are developed with strong protection capabilities against acid attack on teeth.

### SUMMARY OF THE INVENTION

This invention discloses new proteins & peptides & saccharides that have anti-cariogenic capabilities and that are characterized by the presence of one or more components that have the ability to form a complex with calcium ions: such as epsilon-polylysine that is conjugated with one or more bisphosphonyl-, biscarboxyl-, or 3-hydroxy-phthalate-groups or conjugated with casein phosphopeptide, phosvitin or with partially hydrolyzed phosvitin; such as partially hydrolyzed chitosan that is conjugated with one or more bisphosphonyl groups, casein phosphopeptide or with phosvitin or partially hydrolyzed phosvitin; such as bisphosphonylated and biscarboxylated proteins with at least 40% of amino acids consisting of lysine and a molecular weight of above 2 kD (2000 dalton) and such as polymerized casein phosphopeptide and partially hydrolyzed phosvitin.

Especially basic polymers that are conjugated with bisphosphonyl-groups, such as bisphosphonylated epsilon-polylysine, are strong protectors due to the simultaneous presence in one molecule of strong calcium-complexing- and strong acid buffering components. In addition, polylysines, such as epsilon-polylysine have demonstrated antibacterial activity against a large variety of oral cavity bacteria including acid producing bacteria. It indicates it's relevance for the protection of teeth in particular and for control of the bacterial flora in the oral cavity in general.

The products can be used in formulations to protect teeth and to treat the oral cavity: toothpastes, gels, mouth rinses, artificial saliva's . . . for patients and healthy consumers. They have an attractive toxicological profile compared to fluoride, and can be used in food systems; they act additionally to the action of fluoride. The use in combination with fluoride provides excellent and enhanced protection at minimum fluoride dosage.

The invention encompasses competent protein & peptide & saccharide structures, based on in-vitro and in-vivo experiments, as well as production procedures and application conditions.

### BRIEF DESCRIPTION OF THE FIGURES

FIG. 1 provides a schematic representation of homo- and copolymerisation of peptides or proteins with a carbodiimide (EDAC).

FIG. 2 provides a schematic representation of the reaction between epsilon polylysine and a bisphosphonylated epoxide into bisphosphonylated e-polylysine. The epoxide is made from vinylidene diphosphonate.

FIG. 3: The reaction between epsilon-polylysine and 3-hydroxy-phthalic anhydride into 3-hydroxy-phthalated epsilon-polylysine.

FIG. 4: the susceptibility of anaerobic organisms that have been isolated from the oral cavity in the presence of the products (22; bisphosphonylated epsilon-polylysine), (6; epsilon-polylysine),(10; casein phosphopeptide epsilon-polylysine copolymer) or (27; 3-hydroxy-phthalated epsilon-polylysine). The minimum inhibitory concentration (MIC) is given in mg/ml within an explored range of 0.2 to =>3.5 mg/ml.

FIG. 5 The susceptibility of microaerophilic organisms that have been isolated in the oral cavity in the presence of the products (22), (6), (10)or (27) and expressed in MIC.

FIG. 6: The susceptibility of fungi (yeast alike) that have been isolated in the oral cavity in the presence of the products (22), (6), (10) or (27) and expressed in MIC.

FIG. 7: The susceptibility of aerobic organisms that have been isolated in the oral cavity and of standard strains in the presence of the products (22), (6), (10) or (27) and expressed in MIC.

FIG. 8: The susceptibility of Streptococcus spp. strains that have been isolated in the oral cavity and of a standard strain in the presence of the products (22), (6), (10) or (27) and expressed in MIC.

FIG. 9: the Knoop experiment: depth of penetration (y-axis=μm) of a needle in the tooth surface after having protected the tooth surface with nothing (no symbol) or with 0.1% NaF(IV), 7.1% natural phosvitin(3), 7.3% partially hydrolyzed phosvitin(5), 3.6% 3-hydroxy-phthalated e-polylysine(27) or with 1.5% bisphosphonylated epsilon-polylysine(25), after which the surface is also treated with 0.1N acetic acid. From the data a log regression line is calculated. The protection procedure and the treatment with acid is repeated four times (Procedure: see Examples, Methods & Materials, Determination of PF-value). The formulations of the peptide containing solutions are given under “Examples C.Results; in-vitro experiments”. X-axis: 1=penetration in surface of untreated tooth sample; 2,3,4,5=penetration after 4 consecutive treatments with a protector formulation and with acid.

FIG. 10: the Knoop experiment: depth of penetration (μm) of a needle in the tooth surface after having protected the tooth surface with 6.5% casein phosphopeptide (1)(CPP), 7.3% polymerized casein phosphopeptide (CPP)ₙ (2), 3.6% or 7.8% casein phosphopeptide x e-polylysine copolymer (CPP x elys)ₙ resp. (7) and (10), or with 3.6% (CPP x elys)ₙ+NaF mixture (12), and after which the surface of the tooth is also treated with 0.1N acetic acid. From the data a regression line is calculated. The protection procedure and the treatment with acid is repeated four times (Procedure: see Examples, Methods & Materials, Determination of PF-value). The formulations of the peptide containing solutions are given under “Examples C.Results; in-vitro experiments”. X-axis: 1=penetration in surface of untreated tooth sample; 2,3,4,5=peneration after 4 consecutive treatments with a protector formulation and with acid.

### DESCRIPTION OF THE INVENTION

We have, surprisingly, found that the attachment of components that can form a complex with calcium, to basic proteins, peptides or saccharides, results in new molecules that have the ability to protect teeth. They are assumed to accumulate on the tooth surface, to be able to buffer locally and to allow building blocks, such as calcium and phosphate ions, to carry out repair work, according to the principles that have been outlined in chapter “Background of the invention”, part 4) and 5). This protective effect has been demonstrated with a variety of different components that have the ability to complex with calcium, such as: bisphosphonyl- and 3-hydroxy-phthalate groups, casein phosphopeptide (CPP) and partially hydrolyzed phosvitin (Phos-h). In fact, all basic proteins, peptides and saccharides that have been conjugated to such calcium complexing components, demonstrate tooth protection competence.

The attachment of other calcium complexing components such as biscarboxyl groups or phosvitin, to basic proteins, peptides and saccharides, equally provide polymers with protection competence. In many cases the components with the ability to form a complex with calcium, have been conjugated to epsilon-polylysine, that consists exclusively of lysine amino acids, and therefore, provides strong buffering activity. However, other proteins and peptides that contain a variety of amino acid types, can be used, as long as there are enough basic amino acids available (>40%). There is no need for a protein that exclusively consists of lysine. It is also possible to use peptides and proteins that contain the basic amino acid guanidine instead of the lysine amino acid (or a mixture of both). Alternatively, basic saccharide polymers such as water-soluble hydrolyzed chitosan can be used. Most of these products have not been described and no appropriate production procedures have been disclosed. These latter products include: bisphosphonylated epsilon-polylysine (Bispho x elys), casein phosphopeptide epsilon-polylysine copolymer (CPP x elys)ₙ, 3-hydroxy-phthalated epsilon-polysine, hydrolyzed phosvitin epsilon-polylysine copolymer (Phos-h x elys)ₙ and casein phosphopeptide hydrolyzed chitosan copolymer (CPP x hy-chit)ₙ.

In vitro experiments demonstrate that such products have superior protecting competence in comparison to the known alternatives such as casein phosphopeptide and phosvitin.

The ability to protect is a result of the presence of a number of different components in the polymer that merge four properties in one molecule: components that form a strong complex with calcium ions, stimulating the accumulation of the polymer on the tooth surface and assuring that the polymer is at the location where it should be active; the same calcium complexing components prevent the precipitation of calcium and phosphate ions and keep them available for repair work; the presence of a basic polymer backbone enhances the buffering capability, and a residual antibacterial activity disfavors the growth of bacteria. The new products belong to the class of proteins, peptides and saccharides. For this, they are biodegradable and have a better safety profile than prior art polymers based on carbon, such as polyethyleneimine.

In another aspect of the invention we have found that the protection competence of CPP and phosvitin can be improved by changing the size of the molecule. Increasing the molecular weight thermodynamically disfavors dissolution. We have found that the polymerization of CPP (molecular weight: approx. 2000 dalton) is increasing the protection capabilities. This comes with the finding that small sized amines such as Tris (tris-(hydroxymethyl)aminoethane) is less effective than the larger epsilon-polylysine at identical amine concentration. Equally, the hydrolyses of phosvitin (Mw 35000) into peptides with a molecular weight of 3000 dalton results in the disappearance of protection capabilities. If the reduction in molecular weight is modest, other factors, such as remodeling of the three dimensional structure of the protein, may play a role. We have surprisingly found that the hydrolyses of phosvitin with trypsin, thereby effectively removing 15% of the protein end, improves the protection ability substantially.

In another aspect of the invention we have demonstrated that the poly-cationic peptide, epsilon-polylysine, itself provides protection competence as well as substantial antibacterial activity against a large variety of bacteria that reside in the oral cavity, including streptococcus mutans. It can be used as such or in combination with other protecting ingredients in formulations to protect teeth and to treat the oral cavity. Epsilon-polylysine can be replaced by polylysine.

The capacity to protect teeth against caries can be measured with a technique well known in the art (U.S. Pat. No. 5,279,814). Demineralization with acid, decreases the hardness of the tooth surface. The hardness can be quantified indirectly by measuring the depth of penetration of a needle in the surface (measurement of micro hardness). With this procedure, so called “Knoop” method, the needle is penetrating the surface under the pressure of constant weight and leaves a mark on the surface that allows the calculation of the depth of penetration. The more the tooth is suffering from demineralization, the softer its surface will get and the larger the depth of penetration will be (Knoop F., Peters C. G., Emerson W. B., A Sensitive Pyramidal Diamond Tool for Identation Measurments, J. Res. Natn. Bur. Stand. 23, 39-61 (1939).

The procedure includes the determination of the hardness of a human tooth sample with the Knoop method. Subsequently, the tooth is treated with an experimental protecting product outside the oral cavity.

It is cleaned softly with a paper towel until the coating is invisible.

Subsequently the tooth sample is submerged in a batch of human saliva in order to simulate the conditions in the oral cavity. After removal from the saliva batch (after 30 min.) it is treated with acid over a constant time period of 30 min. at 37° C. The acid is washed away, the tooth is dried on air and the hardness is measured again. The change in hardness (delta P in μm) is obtained by comparing with the hardness of the tooth before treatment. This experiment is repeated on other tooth samples. The effect of acid on unprotected teeth is obtained by carrying out the same procedure but without the use of an experimental protecting product (delta Pa). The protecting capacity (“PF” protection factor) of an experimental protecting compound can be defined as:

PF=100−delta P*100/delta Pa

(PF of product X=35: a tooth that is protected with product X exhibits 35% less decrease in hardness when subjected to acid, in comparison to a tooth without a protection coating).

The Products: Phospho-Peptides with Calcium Complexing Components

In one aspect of the invention, we show that casein phosphopeptide that has been polymerized with a carbodiimide (CPP)ₙ, provides more protection compared to non-polymerized casein phosphopeptide (CPP).

The increase in molecular weight and/or in the number of available calcium complexing components per molecule, improves the attachment to the tooth surface.

This is confirmed by the finding that hydrolyzed phosvitin (PPP; Mw 1-3 kD) provides less protection competence compared to natural phosvitin (Mw>30 kD). (CPP)ₙ can be used at a concentration of 0.01% to 30% (preferably between 1 and 10%; on weight basis) in the end formulation that is used for the treatment of teeth. CPP is well soluble in water and the polymerization reaction can be carried out at any concentration of CPP as long as it is dissolved (preferably between 5% and 30% and more preferably between 8% and 20%); the preferred ratio CPP/carbodiimide is between 1/1.5 and 1/0.05 on weight basis and the pH is between 5 and 9. The preferred carbodiimide in water is 1-ethyl-3-(3-dimethylaminopropyl)carbodiimide. When selecting the -preferred dosage of carbodiimide, over-crosslinking, at which the product may partly become insoluble, should be avoided. Alternatively, carbodiimides (e.g. cyclohexylcarbodiimide) can be used that are soluble in organic solvents (DMSO . . . ). In principle the reaction can also be carried out with other known coupling agents such as dimethyl suberimidate, dimethyl pimelimidate, diethyl malonimidate, dimethyl adipimidate, bisepoxides, bis-isocyanates, glutaraldehyde, . . . .

The temperature of the reaction will be between 0 ° C. and 60° C. (preferably between 15 ° C. and 30° C.).

In another aspect of the invention, we disclose that phosvitin which has been partially hydrolyzed with the trypsin enzyme (Phos-h; Mw 22 to 30 kD) provides 25% more protection competence in comparison to natural phosvitin. The enzyme cuts one end of the protein (amino acid 1 to 48 on the amine side), resulting in the removal of a relative hydrophobic piece. The remaining part, which still is relatively large (>25 kD), consists almost exclusively of hydrophilic phosphorylated serine amino acids and may take a three dimensional conformation which is different from the one of natural phosvitin. This may alter its capability to attach to the tooth surface.

Partially hydrolyzed phosvitin (Phos-h) can be used at a concentration of 0.01% to 30% (preferably between 1 and 10%) in the end formulation that is used to treat teeth. The hydrolyses of phosvitin can be carried out in water at a pH between 5 to 9 (preferably 8) with a trypsin/substrate ratio of 0.1% to 50% (preferably 0.5% to 10%), and at a temperature between 0° C. and 70° C. (preferably between 20° C. and 40° C.). Alternatively, chymotrypsin can be used to substitute trypsin and an additional treatment with pepsin at pH 2 to 7 (preferably between 2 and 3) can be carried out. (Phos-h) may contain the 28 kD segment of phosvitin (Gln49-Arg212) that has been described in the literature (A. Goulas et al, Journal of Protein Chemistry, vol 15, no 1, 1-9, 1996).

In general, these data demonstrate that protection capabilities are improved if phosphopeptides of enlarged size are used and if the presence of hydrophobic amino acids is avoided. For this, it is justified to polymerize CPP and to partially hydrolyze phosvitin.

The Products: Amino-Polymers with Calcium Complexing Components

These new compounds are unique as they combine strong attachment capabilities. to the tooth surface with strong buffering capabilities.

They demonstrate a high level of protection competence. The presence of both, amino-groups and components that form a complex with calcium, provide the required combination of properties.

In one aspect of the innovation, it was discovered that the copolymer of casein phosphopeptide with epsilon-polylysine (PF-value of 7.8% (CPP x elys)ₙ: 94) is providing more protection in comparison to polymerised casein phosphopeptide (PF-value of 7.2% (CPP)ₙ: 58). The copolymerisation is carried out in the same way as with the polymerisation of casein phosphopeptide whereby part of the CPP (1 to 99% and preferably 10 to 30%) is replaced by epsilon-polylysine. This (CPP x elys)ₙ can be used at any concentration that is soluble in water, or at a concentration of 0.01% to 30% (and preferably between 0.5% and 10%) in the final formulation that will be used to treat teeth. It can also be used in combination with fluoride. The protecting effects of both products is additive. 0.1% NaF and a mixture of 3.6% (CPP x elys)ₙ with 0.1% NaF exhibit a PF-value of respectively 61 and 100. It is possible to use epsilon-polylysine at any molecular weight. It is also possible to use a chitosan hydrolysate (Mw<30000 dalton; product 13: PF-value of 2.2% (CPP x hy-chit)ₙ: 63) that is water soluble at neutral and basic pH. The reaction procedures (temperature, concentration, pH) are similar to the one of CPP x epsilon-polylysine copolymer. Optionally, CPP and the carbodiimide can be allowed to react (0.5 minute to several hours) before the polyamine is be added. The end-product of the reaction can be used as such for the determination of the hardness value, or can be purified with gel permeation chromatography, ultrafiltration or other purification methods. Also protamine, a basic polypeptide (80 to 200 kD), protamine hydrolysates, synthetic polylysines (Mw 200 to >1.000.000), or proteins of which the amino acid content contains at least 40% lysine, could be used.

In another aspect of the innovation, partially hydrolyzed phosvitin (Phos-h) was conjugated to epsilon-polylysine to (Phos-h x elys)ₙ. The conjugation can be carried out with glutamine, with a carbodiimide or with both.

The reaction pH is preferably above 8 in order to prevent precipitation. (Phos-h x elys)ₙ can be used at a concentration of 0.01% to 30% in the final formulation. The reaction with glutaminase can be carried out at a temperature between 15 to 80 ° C., preferably between 40 to 60° C. at a enzyme concentration of 0.1% to 10% (on protein weight). (Phos-h) can be replaced with natural phosvitin in the conjugation reaction and this has to be considered as part of the scope of this invention.

In another aspect of the invention, we disclose that bisphosphonylated epsilon-polylysine (Bispho x elys) exhibits highest protection capacity (PF-value of 1.5% (Bispho x elys): 99). The phosphonate groups are attached to one and the same carbon atom, resulting in the formation of a strong complex with calcium ions. When using the preferred reaction for the attachment of the bisphosphonyl groups to epsilon-polylysine, the carbon atom between the two phosphonate groups does contain a hydroxyl-group in addition. It contributes to the complexation with calcium. The (Bispho x elys) can be used at a any concentration or at a concentration of 0.01% to 30% (preferably between 0.5% and 10%) in the end formulation that will be used to treat teeth. The product can be made from epsilon-polylysine and epoxy-ethane-diphosphonate (in salt or acid form) in water, alcohols, liquid salts or mixtures of water and alcohol (or other water soluble organic solvents such as aceton, dioxane . . . ), at a temperature of 20 to 90° C. (preferably 40 to 60 ° C.), with a any molar lysine/epoxy ratio preferably between 0.5 to 20. The reaction can be carried out at a pH of 3 to 9, preferably at pH 3 to 5.

A lewis acid catalyst, such as BF3, can be used with a ratio to epoxide of between 0.005 and 10% w/w. In another aspect of the invention it appears that a larger number of bisphosphonyl groups can be attached to denaturated epsilon-polylysine in comparison to non-denaturated epsilon-polylysine (ref product 24: 6.3.bisphosphonyl groups per molecule). Attachment to denaturated epsilon-polylysine results in products with the highest PF-values (the PF-factor of product made with 1.5% non-denaturated polylysine, no. 22, and with denaturated polylysine, no. 24, are respectively 70.4 and 88.9). The urea that is used for the denaturation process can be used at any concentration that is effective but should preferably be used at an elevated dosage (preferably at or above 8M).

The epoxide can be produced, according to a known process (U.S. Pat. No. 3,940,436), from the salt of vinylidene diphosphonate (or the acid form) and hydrogen peroxide (or another oxidation agent) in the presence of a catalyst such a sodium tungstate. In principle it is possible to carry out the production of the epoxide and the attachment to the amino-polymer in one reaction (product 17 and 18); alternatively (product 19 to 23) the epoxide is produced separately and the excess hydrogen peroxide is removed before addition of the amino-polymer. The excess of hydrogen peroxide can be removed by the precipitation of the epoxide; for example with the addition of aceton to the aqueous solution. Both, the one- and two-step reactions deliver a product with a higher PF-value in comparison to epsilon-polylysine itself. The two-step procedure provides products with higher PF-value in comparison to products made according to the one-step procedure: PF-value of 3.6% epsilon-polylysine (product 6): 73; PF-value of 3.5% bisphosphonylated epsilon-polylysine (product 17, 1-step procedure): 82; PF-value of 3.5% bisphosphonylated epsilon-polylysine (product 20; 2-step-procedure): 100.

Despite extensive ultrafiltration of the bisphosphonylated e-polylysine reaction mixture, some phosphonate groups may still be present in the free form, unattached to e-polylysine; such mixture is to be understood as within the scope of this invention.

Another possible procedure for the attachment of bisphosphonates to epsilon-polysine could be taking advantage of a diazomethylene-diphosphonate ester that is produced with a methylene diphosphonate ester, t-BuOK and tosylazide (U.S. Pat. No. 4,150,223). The diazocompound can be transformed in carbonyldiphosphonate ester (U.S. Pat. No. 6,147,245) and reductively aminated with epsilon-polylysine and a reduction agent such as sodium borohydride.

In another aspect of the invention, we describe that 3-hydroxy-phthalated epsilon-polylysine (product 27) exhibits good protection (PF-value of 3.6% (hphth x elys): 90.7), superior to the one of epsilon-polylysine; 3-hydroxy-phthalates are known to complex with calcium. (hphth x elys) Can be made in DMSO, water or water/alcohol mixtures; the ratio 3-hydroxy-phthalic anhydride/polylysine is preferably 0.05 to 1; preferably the anhydride is added to the reaction at 0-6° C. after which the reaction is allowed to continue overnight at 20° C. 3-Hydroxy-phthalated epsilon-polylysine can be used at a concentration between 0.01% and 30% (preferably between 0.5% and 10%) in the endformulation that is used to treat teeth. It is soluble in water at a pH above 9.

Epsilon-polylysine is not the only amino-polymer that can be used. The findings indicate that also polylysine and proteins of which the amino acid content does not consists exclusively of lysine, but of which the amino acid content contains at least 40% lysine, can be used for attachment to components that form a complex with calcium ions (eg. bisphosphonylation . . . ) in order to produce compounds with the ability to protect teeth. Preferably the new products should be larger than the existing ones in the prior art (such as CPP, arginine, small arginine containing peptides with 2-4 amino acids) and the molecular weight should preferably be above 2.5 kD. Arginine or ornithine could be used as alternative to lysine amino acids in the basic proteins. From the results with product 13, 14 and 15 it can be concluded that epsilon-polylysine can be replaced by partially hydrolyzed chitosan (water soluble at neutral and basic pH) in the chemical reactions with components that form a complex with calcium ions. The molecular weight of hydrolyzed chitosan is preferably below 30 kD.

In another aspect of the invention is has been demonstrated that the new protectors are also effective in viscous or gelled formulations (chapter: Examples; C. Results; C1. In vitro-experiments; experiment 4). In the presence of sodium fluoride, it's protection ability is additive to the one of fluoride, enhancing the overall protecting competence of the gel. Hydroxyethylcellulose has been used as a thickener, however another thickener, well known on the market, could be used. It is also possible to replace a traditional thickener such as hydroxyethylcellulose with CPP-epsilon-polylysine copolymer; it is itself both a tooth protector and a creamy thickener as opposed to bisphosphonate e-polylysine that does not enhance the viscosity.

The gelled formulation that contains 7% bisphosphonate epsilon-polylysine and 0.3% sodium fluoride has a PF-value of 93.2, in comparison to a gel with 0.3% sodium fluoride only having a PF of 68%.

### The Products: Amino-Polymers

In another aspect of the invention it was shown that epsilon-polylysine itself has protecting competence (PF-value of 3.6% epsilon-polylysine: 70).

The importance of using products of elevated size is confirmed.

Tris(hydroxymethyl)aminoethane (PF-value 3.4% Tris:35) exhibits less protection ability in comparison to epsilon-polylysine at equal amino content. Epsilon-polylysine provides advantages over the amino-based products known in the prior art: it is larger than arginine and arginine based peptides (with only 1 to 4 amino acids) and it is safer in comparison to polyethyleneimine (PEI). Epsilon-polylysine has obtained the GRAS (Generally Recognized as Safe) status from the FDA and it's use in food is allowed but it is not known as a tooth protecting agent. It can be used at any concentration, preferably between 0.01% to 40%. It can be produced by fermentation from Streptomyces Albulus. Alternatively also synthetic polylysine can be used or protamine or hydrolyzed chitosan (soluble in water at neutral and basic pH; molecular weight below 30000 dalton).

### Antibacterial Activity of Epsilon-Polylysine and Modified E-Polylysine

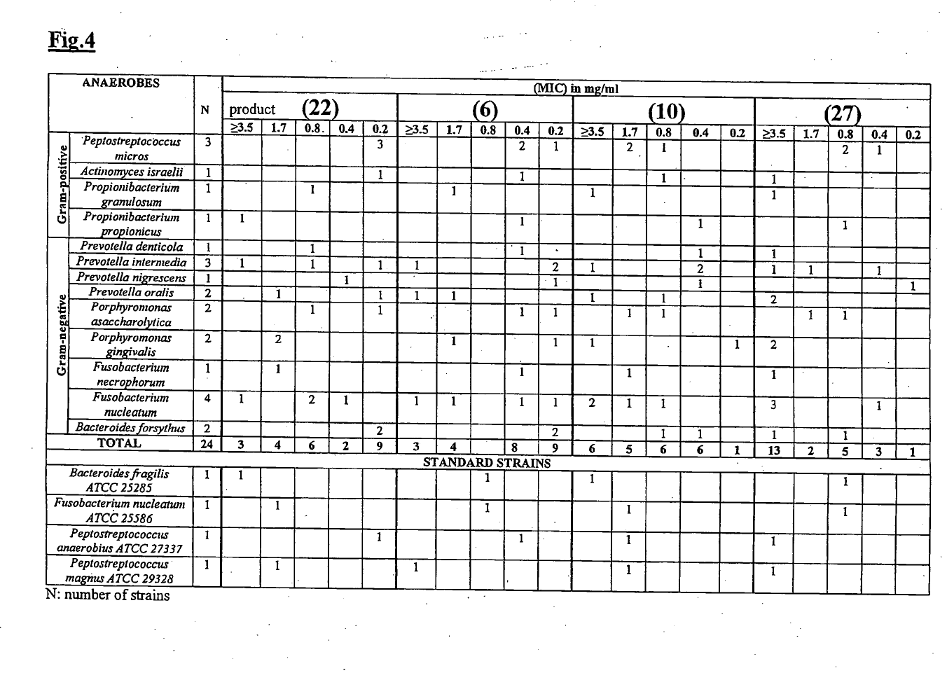

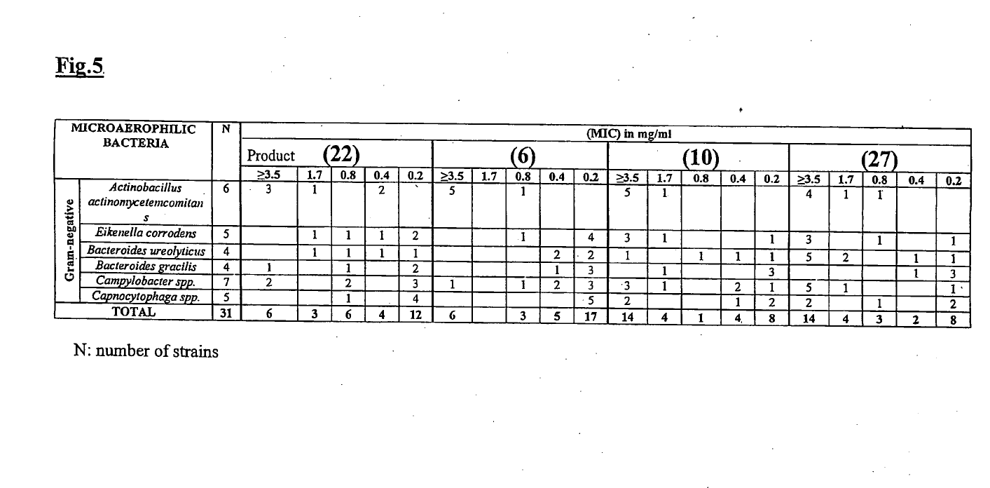

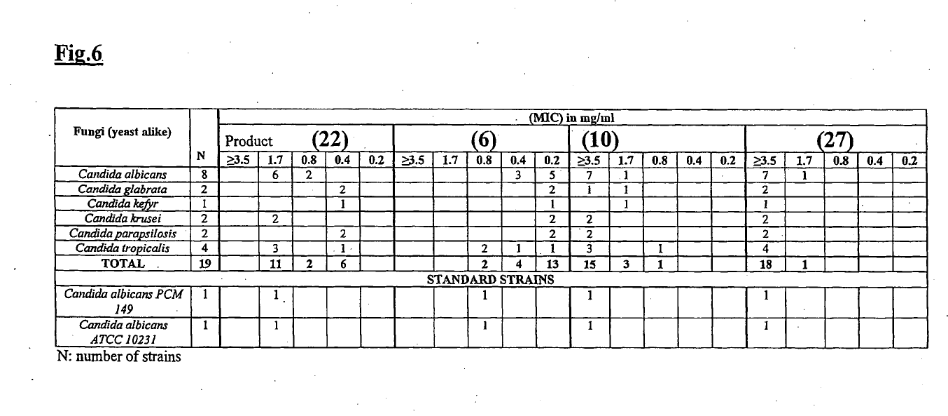

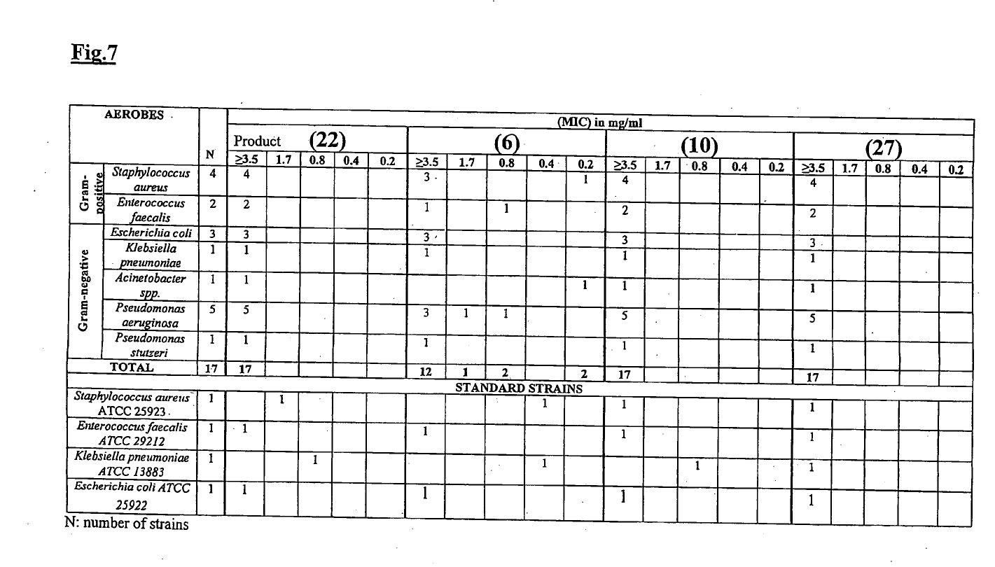

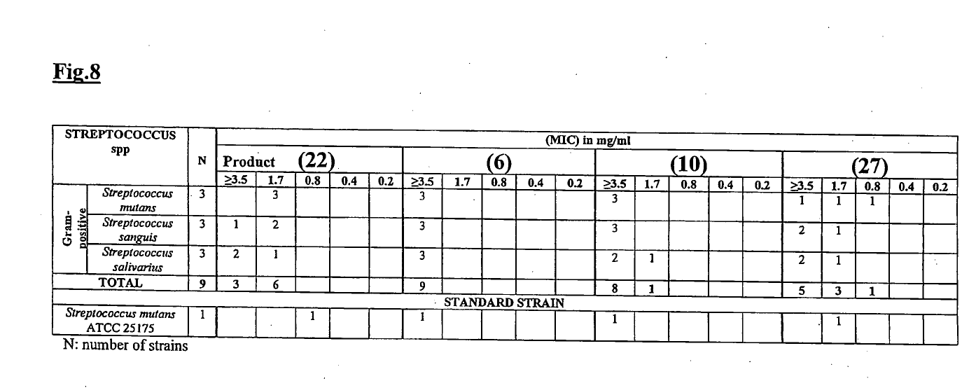

FIGS. 4 to 8.

In another aspect of the invention we have demonstrated that epsilon-polylysine, bisphosphonylated epsilon-polylysine, 3-hydroxy-phthalated epsilon-polylysine and the copolymer of casein phosphopeptide and epsilon-polylysine (CPP x elys)n exhibit antimicrobial activity against bacteria that are residing in the oral cavity. More than hundred different anaerobic-, aerobic-, microaerophilic bacteria and fungi, harvested all from the oral cavity, have been tested.

Epsilon-polylysine exhibits the highest activity against anaerobes. From 28 anaerobic strains the growth of 24 was inhibited by concentrations in the range of 3.5-0.2 mg/ml. The gram-negative bacteria are often susceptible to a low mic-value (minimum inhibitory concentration) of =<0.2 mg/ml, including strains of species Prevotella nigrescens, Poiphyromonas asaccharolytica, Porphyromonas gingivalis, Fusobacterium nucleatum. These bacteria often take part in periodontitis, infections of dental pulp and other infections in the oral cavity. Also the gram-positive anaerobes are often susceptible to low mic-values (=<0.2 to 0.4 mg/ml) of epsilon-polylysine. Bisphosphonylated e-polylysine demonstrated also good activity against anaerobes, but casein phophopeptide e-polylysine and 3-hydroxy-phthalated e-polylysine where less effective.

Bisphosphonylated- and 3-hydroxy-phthalated e-polylysine and (CPP x elys)ₙ are not active against aerobes, but e-polylysine inhibits the growth of 7 strains among 21 in the range of 1.7 to 0.2 mg/ml. Bisphosphonylated e-polylysine inhibits 6 from 9 Streptococcus strains at 1.7 mg/ml.

E-polylysine exhibits the strongest activity against microaerophilic bacteria with an inhibitory concentration of 0.2 mg/ml in the case of 17 from 31 strains.

Bisphosphonylated e-polylysine too, exhibits activity (mic value of 0.2 mg/ml for 12 strains from 31).

Once again e-polylysine was the most active against the tested fungi with an inhibitory concentration of between 0.2 and 0.8 mg/ml for all 21 tested strains.

The antibacterial activity of e-polylysine exhibits multiple activities in the oral cavity at the same time and addresses multiple needs. It does not only contribute to the protection of teeth but also keeps the bacterial flora under control (in a similar way to what histatine, a natural peptide in saliva, does) and also helps to avoid bad mouth odor (halitosis). Indeed, many of the anaerobes that are causing infections such as parodontitis are also contributing to bad mouth odor, by producing sulfur containing compounds. It is in particular relevant to note that at the lowest concentrations (0.2 to 0.4 mg/ml) it exhibits inhibitory activity against 71% of anaerobes that have been harvested from the oral cavity (17 from 24 species) and against 12% of aerobes harvested from the oral cavity (2 from 17). Apart from the use in tooth protection the selective antimicrobial activity of e-polylysine makes it a valuable tool for use against halitosis (bad mouth odor) too. E-polylysine is capable of attacking the organisms responsible for halitosis in a partially selective way.

The strong antibacterial activity (especially also against fungi) makes it a good candidate for use in artificial saliva for patients with Xerostomia (dry mouth). This includes patients with oral cancer, Hodgkin's disease, Sjögren syndrome, HIV . . . . They are often confronted with fungal infection in the oral cavity. Epsilon-polylysine is not only beneficial for it's contribution to the protection of teeth but also for the protection of the oral cavity as a whole.

### In-Vivo Experiments

In-vivo experiments have been carried in order to evaluate under the natural conditions in the oral cavity. The procedures are well known in the art of evaluating tooth protection competence.

A device is constructed that can contain up to 6 experimental tooth samples for positioning in the oral cavity. The device is made of self-curing methacrylate resin based on a plaster cast of the lower jaw of volunteers and fits precisely behind the lowerjaw (P. Bottenberg et al, Clin Oral Invest (2000) 4:153-156). The construction of the experimental tooths samples is outlined in chapter C.2.“Results; in-vivo experiments”. In the oral cavity, the tooth samples are positioned in the device in such a way that the enamel surfaces are subjected to physical contact with the tongue and are submerged in saliva. It is known that the depth of penetration (μm) of a needle in the surface of clean teeth that have resided a few days in the oral cavity, increases (P. Bottenberg et al, Clin; Oral. Invest (2000) 4:153-156). This may be the effect of the local microflora. Pretreatment of the teeth with fluoride before positioning in the oral cavity limits such increase. We have evaluated the protection capacity of experimental proteins & peptides and saccharides by treating the teeth with such product prior to positioning them in the oral cavity and by comparing the penetration depth after a residence time of a five days with the one of the blanco and of tooth samples that have been treated with fluoride instead. In experiments where the device is residing a long time period in the oral cavity (5 days and more), a metal network is positioned just above the surface of the experimental teeth, in order to provide support for the bacteria and stimulate it's growth path. It is also possible to measure the residual protection competence by removing the tooth samples from the oral cavity at the end of the selected residence time and treating them with acid under laboratory conditions. If the saliva and the physical contact with the tongue did not remove the protective agent from the tooth surface, residual protection competence will be measured in comparison to the blanco.

In order to assess the protection ability of experimental products on teeth of patients with oral cancer that are subjected to head & neck radiation, 72 radiated and sterilized tooth samples have been treated with a solution of either 0.1% NaF or with a mixture of 4.2% bisphosphonylated e-polylysine, CaCl₂ 2H₂O and KH₂PO₄ or as a blanco (24 samples for each of the three solutions; 4 volunteers; ref. chapter: Examples C.2.1.). The device was positioned after the lower jaw in the oral cavity for a period of five days. After removal from the oral cavity, the samples where cleaned and the hardness was measured. The depth of penetration of the needle (ref. Knoop method) increased on average 3.7 μm, 1.8 μm and 1.1 μm for tooth samples that have been treated respectively as a blanco, with 0.1% NaF or with 4.2% bisphosphonylated epsilon-polylysine. The in-vivo protection competence is further underlined by the p-values from the statistical Mann-Whithey test and are respectively 0.001 and 0.000 for 0.1% NaF and for 4.2% bisphosphonylated e-polylysine versus the blanco.

A number of samples have been treated in addition with a drop of acetic acid (0. 1N, 30 min., 37° C.) in order to assess the residual protection competence left after having resided in the oral cavity for a period of five days (day and night). The depth of penetration of the needle increased with 10.1 μm, 6.5 μm and 3.9 μm for teeth treated respectively as blanco, with 0.1% NaF or with 4.2% bisphosphonylated e-polylysine. The protection competence of the latter is further confirmed with a p-value of 0.032 (Mann-Whithey) versus blanco.

In another experiment, 126 sterilized tooth samples where treated with a variety of peptides, fixed to the device and positioned after the lower jaw in the oral cavity of 5 volunteers. The device remained in the oral cavity for a period of 12 hours after which the teeth where removed, cleaned and subjected to a drop of acetic acid (0.1N, 30 min., 37° C). Subsequently the acid was removed, the teeth where cleaned and the hardness was measured (Knoop method). The depth of penetration increased 8.82 μm, 5.40 μm, 5.32 μm and 3.77 μm respectively for teeth treated as blanco with 4.2% casein phosphopeptide e-polylysine copolymer, 7.5% phosvitin and with 4.2% bisphosphonylated e-polylysine. The p-values are respectively 0.000, 0.000 and 0.004 for casein phosphopeptide e-polylysine copolymer, bisphosphonylated e-polylysine and phosvitin.

Bisphosphonylated e-polylysine is the stronger protector. Submergence of the tooth sample in a saliva bath prior to adding bisphosphonylated e-polylysine to the surface does not reduce it's protection competence (ref. chapter “examples C.2.2.).

### Other Ingredients in End Formulations

The new tooth protecting polymers can be used with other known ingredients in end formulations for use in the oral cavity: toothpastes, mouth refreshing solutions, mouth rinses, mouth sprays, gels, chewing gum, candies and other food systems, artificial saliva and medical products for the treatment of teeth from patients with oral cancer, Hodgkin's disease,Sjögren syndrome, xerostomia. An overview of such end formulation is provided in U.S. Pat. No. 6,238,648.

The end formulations can contain other components for the protection against caries: fluorides (U.S. Pat. No. 2,946,725 and U.S. Pat. No. 3,678,154; for example sodium fluoride, sodium monofluorophosphate and stannous fluoride or encapsulated fluoride ingredients (for protection against deactivating components such as calcium or orthophosphates). Fluorides are used at a concentration between 0.1% to 1% w/w, preferably between 0.25 and 0.5% on weight basis. The end formulation can contain also other protecting compounds such as those that are mentioned under the chapter “Field of Invention”, including ingredients with antibacterial activity (part 1. natural bactericides, synthetic bactericides, plant extracts, peptides with immunological activity, antibodies against S. Mutans, bacteriophages), sugars to reduce the production of acid (part 2. xylitol, erythritol), enzymes (part 3. e.g. glucanases and dextranases) and a vaccin against glucosyltransferase or peptide analogues to the S. Mutans antigen I/II, ingredients for repair work (part 4. calcium, phosphate, casein,, non-denaturated casein, casein hydrolysates (CPP), buffering components such as chitosan, polyethyleneimine fluorofosfaat, arginine and arginine containing peptides (with 2-4 amino acids).

Ingredients such as calcium salts can be encapsulated in order to avoid interference with fluoride during storage. Calcium salts include calcium chloride, calcium acetate, calcium citrate, calcium butylate, calcium lactate, calcium salicylate or another non toxic anorganic or organic calcium salt at a concentration between 0.1% to 5% w/w.

The end formulation can contain non ionic-, anionic-, amphoteric-, cationic- or zwitterionic detergents as described in U.S. Pat. No. 3,988,433, U.S. Pat. No. 4,051,234, U.S. Pat. No. 3,959,458. Non ionic detergents are condensates from hydrophilic alkylene oxide groups with hydrophobic organic components. For example: poloxamers (sold under the name Pluronic), polyoxyethylene sorbitan esters (Tween), polyethylene oxide condensates of alkyl phenols, condensates of ethylene oxide with reaction products from propylene oxide and ethylene diamine, ethylene oxide condensates from aliphatic alcohols, tertiary amine oxides with a long chain, tertiary phosphine oxide with a long chain, dialkylsulfoxides with a long chain and mixtures. Amphoteric detergents are aliphatic secondary and tertiary amines, with an aliphatic chain and with the presence of an anionic group (e.g. carboxylate, sulfonate, sulfate, phoshate, phosphonate). Anionic detergents are salts of alkylsulfates with 8 to 20 carbon atoms ( for example sodium alkyl sulfate) and salts of sulfonated monoglycerides from fatty acids with 8 to 20 carbon atoms. Examples: sodium lauryl sulfate and sodium coconut monoglyceride sulfonate, sarcosinates such as sodium lauroyl sarcosinate, sodium lauryl sulfoacetate, sodium lauroyl isethionate, sodium laureth carboxylate, sodium dodecyl benzenesulfonate or mixtures. Often the dosage of an anionic detergent is between 0.025% to 9% and preferably between 0.1% and 5% W/W.

Thickeners can be use in the end formulation to provide the desired rheological profile: guar gum, carboxyvinyl polymers, carageenan; Konjac, scleroglucan, carboxymethyl cellulose, hydroxyethyl cellulose, polyoxyethylene polyoxypropylene glycol copolymers, gum karaya, gum arabic, gum tragacanth and xanthan in a concentration of 0.1% to 15%. Cross-linked polymers from acrylic acid, such as Carbopol from BF Goodrich are known in the sector.

The end formulation can contain a humidifier. Polyalcohols provide a wet feeling and prevent the product from becoming hard upon contact with air. They include glycerin, sorbitol, butylene glycol, polyethylene glycol, sorbitol.

The end formulation can contain abrasives: silica such as amorphous hydrated silica, sodium metaphosphate, potassium metaphosphate, tri-calcium phosphate, calcium phosphate two hydrate, calcium phosphate, calcium pyrophosphate, sodium bicarbonate, calcium bicarbonate, hydrated alumina. Sometimes are polymers used as abrasive (U.S. Pat. No. 3,070,510): melamines, polyphenols, ureas, urea-formaldehyde . . . : silica based abrasives are described in U.S. Pat. No. 3,538,230 and U.S. Pat. No. 3,862,307 (included for reference). “Syloid” (from W.R. Grace & company) and “Zeodent” (from J.M. Huber corporation) are well known in the field. Often toothpastes contain between 10% and 70% abrasive or a mixture of abrasives.

The end formulation can contain products against tooth-stone such as pyrophosphate salts such as Na.sub.4 P.sub.2 O.sub.7, K.sub.4 P.sub.2 O.sub.7, Na.sub.2 K.sub.2 P.sub.2 O.sub.7, Na.sub.2 H.sub.2 P.sub.2 O.sub.7 and K.sub.2 H.sub.2 P.sub.2 O.sub.7, sodium hexamethaphosphate, sodium tripolyphosphate and cyclic phoshphates such as sodium trimetaphosphate. The dosage is about 0.5% to 10% w/w. Anionic polycarboxylates or carboxylated chitosan could be used eventually in order to increase the anti-tooth stone effect. Copolymers of maleic anhydride with other ethylenic monomers such as methyl vinyl ether with a molecular weight between 30.000 and 1.000.000. and preferably between 30.000 and 500.000 are known under the name Gantrez (U.S. Pat. No. 4,627,977). The concentration in the end formulation is between 0.5% and 5%. Other possibilities include zinc citrate trihydrate, polyphosphates, diphosphonates (EHDP).

The end formulation can contain aroma's, often at a concentration between 0.00% and 5% and preferably between 0.5% and 1.5% w/w. Examples are: spearmint, peppermint, menthol, anethole, methyl salicylate, cassia, 1-menthyl acetate, eugenol, parsley oil, oxanone, alpha-irisone, marjoram, propenyl guaethol, vanillin, thymol, linalool, cinnamaldehyde glycerol acetal, wintergreen, sassfras clove, sage, eucalyptus, marjoram, cinnamon, lemon, lime, grapefruit, orange.

The end formulation can also contain sweeteners; besides the known anticariogenic sweeteners the following products are valuable: sucrose, glucose, saccharin, dextrose, levulose, lactose, mannitol, sorbitol, fructose, maltose, xylitol, saccharin salts, thaumatin, aspartame, D-tryptophan, dihydrochalcone, acesulfame and cyclamate salts.

The end formulation can also contain ingredients against over-sensitivity (for example potassium nitrate or potassium citrate), whitening agents (hydrogen peroxide, calcium peroxide, urea peroxide), preservatives, cooling agents (carboxamides, menthol, ketals), anti-inflammatory ingredients (aspirin, ibuprofen, naproxen . . . ).

The formulation can be provided in a one- or two-stage system. The ingredients of a “two-stage” formulation are stored seperately in two compartments and are mixed just before use.

The compositions of end formulations are known and are added for reference: for example for toothpastes U.S. Pat. No. 3,988,433, for mouthrinses U.S. Pat. No.3,988,433, for candies U.S. Pat. No. 4,083,955, for chewing gum U.S. Pat. No. 4,083,955 and for subgingival treatment U.S. Pat. No. 5,198,220.

Toothpastes and gels often contain an abrasive (10% to 50%), a detergent (0:5% to 10%), a thickener (0.1% to 5%), a humidifier (10% to 55%), an aroma (0.04% to 2%), a sweetener (0.1% to 3%), a coloring agent (0.01% to 0.5%), water (2% to 45%) and eventually an anticariogenic product (0.05% to 10%) or a product against tartar (0.1% to 13%).

Mouth rinses and sprays often contain water (45% to 95%), ethanol (0% to 25%), a humidifier (0% to 50%), a tensio-active agent (0.01% to 7%), an aroma (0.04% to 2%), a sweetener (0.1% to 3%) a coloring agent (0.001% to 0.5%) and eventually an anticariogenic product (fluoride; 0.05% to 0.3%) or a product against tooth stone (0.1% to 3%).

Another formulation concerns non-abrasive gels (subgingival gels). They contain a thickener (0.1% to 20%), a humidifier (0.1% to 910%), an aroma (0.04% to 2%), a sweetener (0.1% to 3%), a coloring agent (0.01% to 0.5%), water (2% to 45%) and an anticariogenic or anti-tooth stone product.

Chewing gum formulations often contain gum (50% to 99%), an aroma (0.4% to 2%), a sweetener (0.01% to 20%) and optionally an anticariogenic product. Candies, mints, capsules, tablets and other food systems have been described in U.S. Pat. No. 4,642,903, U.S. Pat. No. 4,946,684, U.S. Pat. No. 4,305,502, U.S. Pat. No. 4,371,516, U.S. Pat. No. 5,188,825, U.S. Pat. No. 5,215,756, U.S. Pat. No. 5,298,261, U.S. Pat. No. 3,882,228, U.S. Pat. No. 4,687,662, U.S. Pat. No. 4,642,903.

### EXAMPLES

### A. Methods & Materials

Products. Casein phosphopeptide (CPP, Mw 1-2 kD, 1000-2000 dalton), Tris(hydroxymethyl)-aminoethane (Trizma), vinylidene bisphosphonate, chitosan, hydrolysed chitosan (Mw: 2000 to 30000 dalton), 3-hydroxy-phthalic anhydride, 1-(3-(dimethylamino)propyl)-3-ethylcarbodiimide hydrochloride, sodium fluoride, epsilon-polylysine (Mw 4100 dalton), carboxylated chitosan, phosvitin and trypsin are available on the market.

Phosvitin hydrolysates with a molecular weight of 1-3 kD (PPP) can be obtained by partial alkaline dephoshphorylation of phosvitin, followed by enzymatic hydrolyses with trypsin (B. Jiang et al., J. Agric. Food Chem 2000, 48, 990-994).

The partially hydrolysated phosvitin with a molecular weight above 20 kD and that has been used in the in-vivo experiment C.2.2., has been made from the proteins (containing phosvitin) of egg-yellow, that are obtained after extraction on industrial scale of the lipids from egg-yellow. The 1.5 L protein solution (10%) is filtered at pH 8 and 3.75 mg trypsin (Sigma; 10100 units BAEE/mg) is added; the reaction is allowed to proceed overnight (14 h) at 37° C. The pH is adjusted to 2.5 and 112.5 mg pepsin (Merck) is added; the reaction is allowed to proceed 9 hours at 37° C. The pH is adjusted to 7.0 and the solution is desalted by diafiltration with a membrane having a cut off of 10 kD.

The solution is purified on a QAE sepharose column HP. After charging the column, the product is eluted with a salt gradient (50 mM EDTA pH 7.0+7.5 g/L NaCl and 50 mM EDTA pH 7.0+58.44 g/L NaCl. The fraction with hydrolysated phosvitin is desalted on column (G25-Hiprep 26/10 desalting).

### Determination of the PF-Value

The ability of a product to protect teeth (PF-value) is determined with the Knoop method. A tooth sample, size 3×3×3 mm is cut from a human tooth and embedded into polymerizing resin in such a way that the surface of the tooth sample appears on the surface of the (resin) cube after the resin has become solid. The surface of the tooth is mechanically polished with rotating silicon carbide abrasive paper (Struers P1200-P2400-P4000) and the micro hardness of the enamel is determined with a Knoop diamant needle (Leitz-Wetzlar;

microdurometer “Durimet”; weight 50 gr;
penetration-time: 30 seconds (Collys et al., J. Dent. Res. 1990; 69: 458-462)).

The penetration leaves a mark on the tooth surface, which is related to the depth of penetration (μm) and which can be analyzed under the microscope. Healthy teeth exhibit a depth of penetration of 40 to 42 μm. The determination of the depth of penetration is repeated six times on each of the tooth samples and the average value is calculated. In order to determine the effect of acid on the hardness of the surface, use is made of an acetic acid buffer from Merck (pH 5; 0.1N). One drop of acid is positioned on the enamel for a period of 30 minutes at 37° C. The sample is cleaned with demineralized water and dried. The hardness is measured again according to the method of Knoop. This procedure is repeated 4 times, which means that the sample has been subjected to acid for a total period of two hours.

A graph (Y: penetration depth μm; X: 5 treatment stages) can be made on the basis of 5 average values (each of them based on six measurement), one obtained before treatment with acid, and the other 4 obtained after each of the 4 treatment-stages with acid. A logaritmic regressionline is calculated.

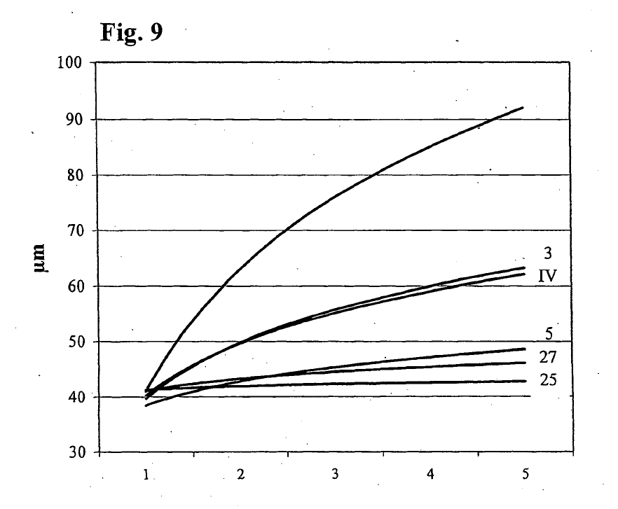

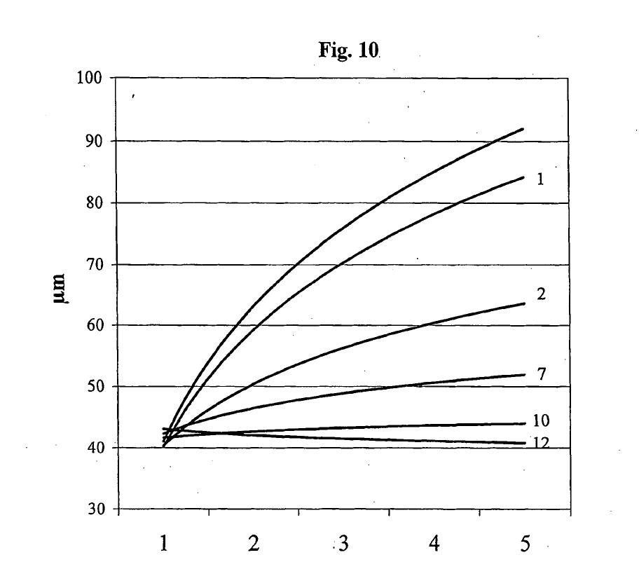

Healthy teeth exhibit a depth of penetration of approximately 95 μm after treatment with acid for a period of two hours (FIGS. 9 and 10).

The evaluation of the protection capacity of an experimental product under in-vitro conditions, is carried out in the following way: the surface of a tooth sample is treated with a drop of experimental coating (30 minutes at 39° C.).; the treatment is repeated one more time after which the drop is removed with air under pressure and the enamel is cleaned softly with a paper towel until there are no visible signs anymore of the coating. Subsequently, the tooth is submerged in a bath of natural saliva for a period of 30 minutes in order to simulate conditions in the oral cavity. The tooth is recovered from the saliva, dried and the enamel is treated with a drop of acid (39° C., 30 minutes). The acid is washed away, the tooth is dried and the hardness is measured six times. The procedure (coating/coating/saliva batch/acid treatment) is carried out four times and overtime the enamel is gradually getting softer. A graph and a logarithmic regression line is made with the data of thirty hardness measurements. The difference between the depth of penetration at the start and the one after 4 treatment-stage with acid is determined (=delta P in μm). In a similar way, the change of depth of penetration resulting from the treatment with acid (4 stages; each 30 minutes) on a tooth sample that has not been protected beforehand with a protecting coating, is determined (=delta Pa).

The ability of an experimental product to protect teeth, is quantified with the PF-value: 100−(delta P)* 100/(delta Pa) (see also chapter “Description of invention”).

The Determination of the Susceptibility of Microorganisms that have been Isolated from the Oral Cavity to Epsilon-Polylysine Bisphosphonylated E-Polylysine Casein Phosphopeptide E-Polylysine Copolymer and 3-Hydroxy-Phthalated E-Polylysin.

The investigations included more than hundred strains isolated from the oral cavity: : anaerobes, microaerophilic bacteria, aerobes, Streptococcus spp. and yeast like fungi. The microorganisms were isolated from saliva, mucosal membrane, gingival pockets and caries decay. The susceptibility (MIC) of bacteria and yeastlike fungi were determined by means of plate dilution technique in agar.

The investigated samples were dissolved in sterile distilled water (immediately before the experiment) to obtain the following concentrations: 3.5, 1.7, 0.8, 0.4 and 0.2 mg/ml and were added into appropriate agar. The plates were inoculated using a Steers multipoint inoculator. Inoculum contained 10⁶ CFU/spot. In each experimental series, the growth of strains on the culture medium without the compounds investigated was checked. The concentration at which no macroscopic growth of the microbes was observed on the medium was regarded as the lowest concentration, inhibiting the growth of microbes (MIC). The examinations of the susceptibility of microbes to the peptides were carried out twice.

Anaerobes. The susceptibility of bacteria was determined by means of the agar dilution methods with Brucella agar supplemented with 5% defibrinated sheep's blood, menadione and hemin. Sample solutions with concentrations from of 3.5-0.2 mg/ml were added into agar. The agar plate without investigated samples was included as a bacterial growth control. The inoculum of 10⁶ CFU/ml was applied to the agar plates with Steers replicator. Incubation was performed for 48 hrs at 37° C. (310° K) in anaerobic jars containing a mixture of 10% CO₂, 10% H₂ and 80% N₂, in the presence of palladium catalyst and indicator of anaerobiosis.
Microaerophilic bacteria. The susceptibility of bacteria was determined by means of the agar dilution methods with Brucella agar supplemented with 5% sheep's blood. Samples solutions with concentrations from of 3.5- 0.2 mg/ml were added into agar. The agar plate without investigated samples was included as a bacterial growth control. The inoculum of 10⁶ CFU/ml was applied to the agar plates with Steers replicator. The incubation was performed at 37° C. in anaerobic jars with Campy Pak (bio Merieux) for 48 hours.
Streptococcus spp. The susceptibility of bacteria was determined by means of the agar dilution methods with Mueller-Hinton agar supplemented with 5% sheep's blood. Samples solutions with concentrations from of 3.5- 0.2 mg/ml were added into agar. The agar plate without investigated samples was included as a bacterial growth control. The inoculum of 10⁶ CFU/ml was applied to the agar plates with Steers replicator. Incubation was performed for 24 hrs at 37° C. (310° K) in anaerobic jars containing a mixture of 10% CO₂ and 90% N₂.
Aerobes. The susceptibility to investigated samples was determined by means of the plate dilution technique in Mueller-Hinton agar. Samples solutions with concentrations from of 3.5- 0.2 mg/ml were added into agar. The agar plate without investigated samples was included as a bacterial growth control. The inoculum of 10⁶ CFU/ml was applied to the agar plates with Steers replicator. Plates were incubated for 24 hrs in 37° C. under aerobic conditions.
Yeast like fungi. The susceptibility of fungi to investigated samples was determined by means of the plate dilution technique in Sabouraud's agar. Samples solutions with concentrations from of 3.5- 0.2 mg/ml were added into agar. The agar plate without investigated samples was included as a fungi growth control. The inoculum of 10⁶ CFU/ml was applied to the agar plates with Steers replicator. Plates were incubated for 24 hrs in 37° C. under aerobic conditions.

### Chemical Analyses & Purification Methods

¹H—, ¹³C—, ³¹P—NMR spectra are recorded on a Bruker AC 250 NMR. The phosphor content is determined with a Thermofinnigan Element II (HR ICPMS). Gel Permeation Chromatography is carried out with Sephadex G-25 fine gel. Ultrafiltration purification procedures are carried out with Millipore prep/scale cartridges from cellulose (cut off: 1 kD) and a Millipore “Easy load masterflex” peristaltic pump.

### B. Production Procedures

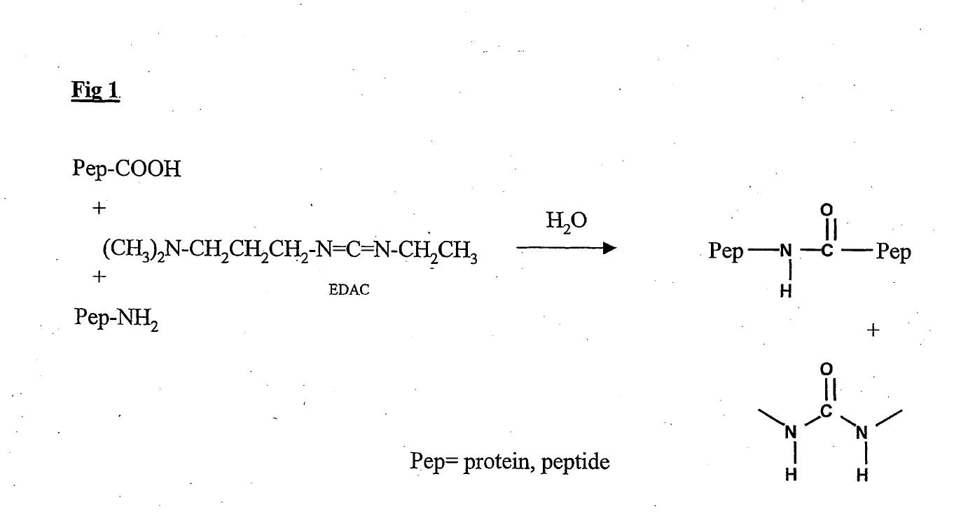

Polymerized Casein Phosphopeptide (CPP)ₙ, (product 2) (FIG. 1).

Add 190 mg casein phosphopeptide to 2.25 gr deionized water. Cool with ice at 6° C. and add 200 mg 1-ethyl-3- (3-dimethylaminopropyl)-carbodiimide (EDAC) and allow to react overnight at 20° C.

Add 735 mg CaCl₂.2H₂O and 408 mg KH₂PO₄ and increase pH to 7 with 8N NaOH. Determine the PF-value.

Partially Hydrolyzed Phosvitin (Mw: 22 to 30 kD), (Phos-h),(Product 5)

Dissolve 200 mg phosvitin in 6 ml water and adjust the pH to 8 with 0.1N NaOH. Add trypsin (0.5% w/w on protein; >10000 BAEE units; Sigma Chemicals Corp.) with incubation time of 5 hours at 37° C. Adjust the pH to 5 to end the reaction. Ultrafiltrate the solution (cut off 10 kD). Acidify the product by passing it over an Amberlite IRC-50 column (acid form) and add CaCl₂ (0.5M) over a period of one hour. Ultrafiltrate on membrane with cut off 10 kD and freeze-dry the product.

(Phos-h) is dissolved in water and mixed with CaCl₂.2H₂O and KH₂PO₄ (the dosages are mentioned in the table “Determination of hardness; experiment 2; product 5” and the PF-value is determined.

Casein Phosphopeptide Epsilon-Polylysine Copolymer(CPP x elys)ₙ; (10)

Dissolve 10 grams of casein phosphopeptide in 86 ml deionized water and cool at 6° C. with ice. Add 1.6 grams EDAC. Add 4.1 grams epsilon-polylysine after 15 minutes. Adjust the pH to 7.8 and allow the reaction to continue overnight (20 hours) at 20° C. (add some additional water, 40ml, in case the viscosity is increasing too much).

Subsequently, add 300 ml deionized water and adjust the pH to 9. Ultrafiltrate with Millipore prep/scale cellulose cartridge with cut off 1000 dalton and produce 2 liter of filtrate; freeze-dry (or spray dry) the retentate.

Dissolve (CPP x elys)ₙ in water and add CaCl₂.2H₂O and KH₂PO₄ (the dosages are mentioned in the table “Determination of hardness; experiment 3; product 10″; and the PF-value is determined.

Casein Phosphopeptide Hydrolyzed Chitosan Copol. (CPP x hy-chit)ₙ;(13)

Dissolve 155 mg casein phosphopeptide in 2.25 gr deionized water. Add 100 mg EDAC and allow to react for one hour at 6° C. Add 35 mg hydrolyzed chitosan (Mw 2000 dalton) and 100 mg EDAC and allow to react overnight at 20° C. Add CaCl₂.2H₂O and KH₂PO₄ according to table “Determination of hardness; example 3; product 13”. Determine the PF-value.

Hydrolyzed Phosvitin x Epsilon-Polylysine Copol. (Phos-h x elys)ₙ; (16)

Dissolve 135 mg phosvitin hydrolysaat (Phos-h) in 1.125 gr deionized water. Add 55 mg epsilon-polylysine and adjust the pH to 8.2. Add 16 mg glutaminase and allow to react one hour at 60° C. Add 145 mg EDAC and allow to react overnight at 20° C. Add CaCl₂.2H₂O and KH₂PO₄ according to table “Determination of hardness; example 3; product 16”. Determine the PF-value.

Bisphosphonylated Epsilon-Polylysine (Bispho x elys) (Product 18);
1-step procedure

Dissolve 165 mg vinylidene bisphosphonate (2-sodium salt; pH 6) in 1 ml hydrogen peroxide (30%); add 6 mg sodium tungstate and 68 mg trifluoro-acetic acid; allow to react for one hour at room temperature. Add 120 mg epsilon-polylysine and allow to react for 5 hours at 50 to 55° C. A product is precipitating during the reaction. Remove the solvent by decantation and dissolve the precipitate in water with addition of NaOH 8N. Purify on a Sephadex 25G column with 0.01N NaOH; freeze-dry the product. Add CaCl₂.2H₂O and KH₂PO₄ according to table “Determination of hardness; example 3;product 18”. Determine the PF-value.

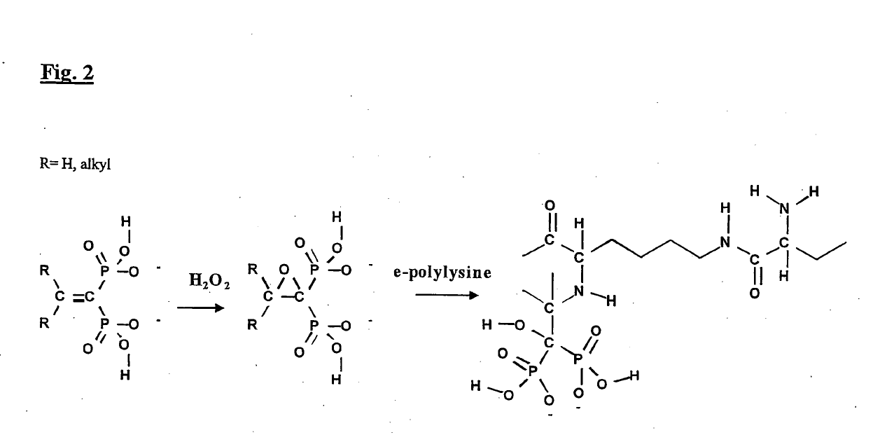

Bisphosphonylated Epsilon-Polylysine (Bispho x elys) (Product 22) (FIG. 2):

2-step procedure (attachment of the bisphosphonate component to non-denaturated e-polylysine in isopropanol/water at acid pH).

### Step 1.

Dissolve 23 grams of vinylidene bisphosphonate (2-sodium salt, solution pH 6) in 85 ml hydrogen peroxide (30%) and add 77 mg sodium tungstate. Allow three hours to react at 60° C. Add 160 ml aceton and an 2nd layer will separate at the bottom; cool and remove the solvent by decantation. Wash the 2nd layer with aceton, remove aceton by decantation and dry.

(Remark: the reaction can also be carried out with vinylidene bisphosphona-te (4-sodium salt, solution pH is basic); removal of aceton/water can be carried out with another known method, other than decantation).

### Step 2.

Add 60 ml water to the dried product of step 1. Add 10 grams epsilon-polylysine and 1.4 ml BF3 (optionally); adjust the pH to 4 with HCl and add 67 ml isopropanol. Allow the reaction to proceed overnight at 50 to 55° C. and adjust the pH afterwards to 6.9 with NaOH 8N. An 2nd layer appears with the addition of 150 ml aceton; cool and separate solvent by decantation. Wash the 2nd layer with aceton. Remove the solvent by decantation and dry. Add 300 ml water and adjust the pH to 9 with NaOH 8N. Ultrafiltrate with Millipore prepscale cartridge (cut off 1000 dalton) with a NaOH solution of 200 mg/liter. Produce 2 liter of filtrate and (freeze) dry the retentate. Add CaCl₂.2H₂O and KH₂PO₄ according to table “Determination of hardness; example 3; product 22”. Determine the PF-value.

¹H-NMR (D₂O): chemical shift (integration): 1.35(2),1.55(2), 1.75(2) en 3.2(2) CH₂ groups of epsilon-polylysine; 3.65 and 2.6(1) CH₁-component of epsilon-polylysine (partially relocated); 3.0 (0.25): new signal, is not present in spectrum of epsilon-polylysine; CHsub2 group from coupled vinylidene component

(chemical shift suggests N—CH₂ coupling and not to O—CH₂ coupling; integration suggests four bisphosphonyl groups per epsilon-polylysine molecule of (+−4000 a 4100 dalton).

Number of bisphosphonyl groups per epsilon-polylysine molecule according to P-determination with HRICPMS: 4.3.

Bisphosphonylated Epsilon-Polylysine (Bispho x elys) (Product 23);
2-step procedure (attachment of the bisphosphonate component to non-denaturated e-polylysine in water at acid pH).

### Step 1.

Add 279 mg vinylidene bisphosphonate (2-sodium salt, solution pH 6) to 1 ml of hydrogen peroxide (30%); add 6 mg sodium tungstate and allow the reaction to proceed for 4 hours at 70° C. Add 2 ml aceton and remove solvent from 2nd layer by decantation and dry under vacuum.

### Step 2.

Add 120 mg epsilon-polylysine, 0.7 ml deionized water and (optionally) BF₃ to the product of step 1 and adjust the pH to 4. Allow the reaction to proceed overnight at 50° C. and adjust the pH afterwards to 8.2. Purify the mixture on Sephadex 25-G column with aqueous NaOH (200 mg/L).

Number of bisphosphonyl groups per epsilon-polylysine molecule according HRICPMS: 3.9.

Add CaCl₂.2H₂O and KH₂PO₄ according to table “Determination of hardness; example 3; product 23”. Determine the PF-value.

Bisphosphonylated Epsilon-Polylysine (Bispho x elys);
2-step procedure (attachment of the bisphosphonate component to denaturated epsilon-polylysine in isopropanol/H2O at acid pH).

### Step 1.

Add 279 mg vinylidene bisphosphonate (2-sodium salt, pH solution 6) to 1 ml hydrogen peroxide (30%); add 6 mg sodium tungstate and allow the reaction to proceed 4 hours at 70° C. Add 2 ml aceton and a 2nd layer appears; remove the solvent by decantation and wash the 2nd layer with aceton, remove the aceton with decantation and dry the product, the epoxide, under vacuum.

### Step 2.

Denaturate 120 mg epsilon-polylysine in 0.7 ml water in the presence of 336 mg urea over a period of 6 hours at 40° C.

Add the denaturated epsilon-polylysine to the product of step 1 the epoxide; Add 1 drop of BF3 optionally and adjust the pH to 4. Add 0.8 ml isopropanol and allow the reaction to proceed overnight at 50° C. and increase the pH afterwards to 6.5. Add aceton, cool and a 2nd layer appears and remove the solvent by decantation; wash the 2nd layer with aceton and remove the solvent by decantation; dry the product under vacuum. Dissolve the 1 ml product in water and purify on a Sephadex 25-G column at pH 11 (with 200 mg/L NaOH in water) and adjust pH to 6.5 afterwards and freeze-dry the product. Purify again on Sephadex G-25 column at pH 6.5.

The number of bisphosphonate groups present per epsilon-polylysine molecule (HICPMS): 11.

Bisphosphonylated Epsilon-Polylysine (Bispho x elys) (Product 24);
2-step procedure (attachment of the bisphosphonate component to denaturated epsilon-polylysine in isopropanol/H2O at acid pH).

### Step 1.

Add 550 mg vinylidene bisphosphonate (2-sodium salt, solution pH 6) to 2 ml hydrogen peroxide and add 16 mg sodium tungstate. Allow the reaction to proceed for 3 hours and 20 minutes at 65° C. and at a pH of 4.6.

Adjust the pH afterwards to 6.6 and add aceton. A 2nd layer appears.

Cool and remove the solvent by decantation; wash the 2nd layer with aceton, remove the aceton by decantation and dry the product.

### Step 2.

Denaturate 240 mg epsilon-polylysine in 1.2 ml water in the presence of 675 mg urea over a period of 7 hours at 40° C. Add the denaturated epsilon-polylysine to the product of step 1, the epoxide, add 2 drops of BF3 optionally and adjust the pH to 4.0. Add 1.35 ml isopropanol and allow the reaction to proceed overnight at 56° C. Adjust the pH afterwards to 6.7 and add aceton. Cool and remove the solvent by decantation. Wash the remaining 2nd layer with aceton, remove the solvent by decantation and dry under vacuum.

Dissolve the product in water, adjust the pH to 8.1 and purify by ultrafiltration (cut-off 1 kD; solvent: 20 mg NaOH/liter); freeze-dry the product.

The number of bisphosphonate groups present per epsilon-polylysine molecule: 6.3.

Bisphosphonylated Epsilon-Polylysine (Bispho x elys) (Product 25).
2-step procedure (attachment of the bisphosphonate component to denaturated epsilon-polylysine in water at acid pH).

### Step 1

Add 20.8 gr vinylidene diphosphonate (2-sodium salt, solution pH 6) to 42 ml hydrogen peroxide (30%) and 250 mg sodium tungstate, adjust the pH to 4.6 and allow to react for 3 hours at 69° C.; adjust the pH afterwards to 6.4.

Add 80 ml aceton (a 2nd layer appears); cool and remove the aceton/water layer by decantation; wash the 2nd layer again with aceton and remove the aceton by decantion; dry the 2nd layer under vacuum optionally.

### Step 2

Denaturate 5 gr epsilon polylysine in 9 ml water in the presence of 10.1 gr urea (4 h, 20° C.). Add the denaturated e-polylysine to the product from step 1 (epoxide), add 0.68 ml BF3 optionally, adjust the pH to 4 and allow the reaction to proceed overnight at 50° C. Afterwards, adjust the pH to 7.0 and ultrafiltrate with an aqueous solution of NaOH 40 mg/L. Produce 2 liters of filtrate and freeze-dry the retentate.

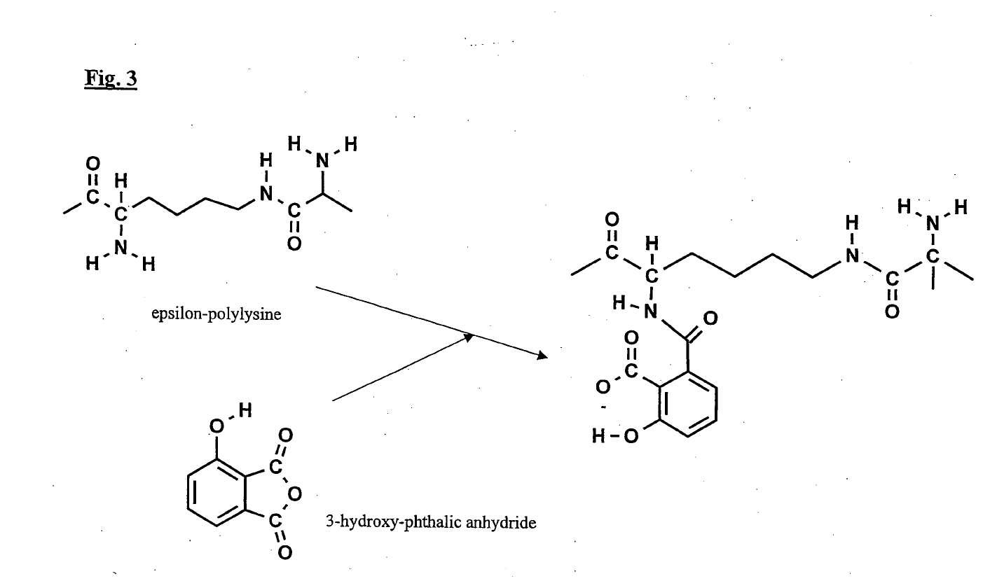

### 3-Hydroxy-Phthalated Epsilon-Polylysine. (Product 27) (FIG. 3).

Add 5.4 grams epsilon-polylysine and 3.2 grams 3-hydroxy-phthalic anhydride to 40 ml water free DMSO at 6° C. A yellow oil appears after one night at 20° C. Wash with aceton, remove the solvent by decantation and dry under vacuum.

Dissolve the product in 300 ml water with the addition of NaOH until the pH is 11. Ultrafiltrate at 35° C. on a Millipore prep scale cellulose cartridge (cut off 1000 dalton) and at a pressure of 1.5 bar with 0.05N NaOH. Produce 2 liter of filtrate and freeze-dry the retentate. Add CaCl₂.2H₂O and KH₂PO₄ according to table “Determination of hardness; example 3; product 27”.

Determine the PF-value.

### C. Results

### C.1. In-Vitro Experiments

The procedure for the determination of the ability of products to protect teeth, is outlined in chapter “Examples; Method & Materials; Determination of the PF-value”. Sterilized tooth samples are treated with a solution that contains a specified dosage of the product under investigation. The delta P and PF-factor is determined according to the procedure.

Measurement of Hardness. Experiment 1: General Products
Code: Productnumber (A), Experimental product (B), delta P μm (C), PF factor (D), % experimental product/water w/w (E), pH (hardness test)(F).

| A | B | C | D | E | F |
|---|---|---|---|---|---|
| I | none/no saliva treatment | 59 | 0 | — | — |
| II | none/with saliva treatment | 59 | 0 | — | — |
| III | sodium phosphate pH 9 | 74.5 | −38 | 3.3 | 9 |
| IV | sodium fluoride (0.1%) | 21 | 61.1 | 0.1 | 7.6 |
| V | Tris(hydroxymethyl)aminoethane | 35.1 | 35 | 3.4 | 9 |

### Remark:

delta P: the difference in the depth of penetration of a needle in the tooth surface (μm) before and after four treatments; each treatment consists of three stages: pre-treatment of the surface with an experimental product, submergence in a saliva batch and treatment with acetic acid.
Delta Pa: the difference in the depth of penetration of a needle in the tooth surface before and after four treatments with acetic acid ( no use of an experimental product, no treatment with saliva).
PF-factor: 100−(delta P)*100/(delta Pa); example: a PF-factor of 60 for product “x” means that 60% of the reduction in hardness of the tooth surface that is caused from the treatment with acid, can be avoided by pre-treating the tooth surface with product “x”.

As for experiment II, no use is made of an experimental product, but the tooth sample is subjected to a saliva batch (for a period of 30 minutes, as is also the case for III, IV and V) prior to each treatment with acid. Alkaline sodium phosphate (III) does not protect but sodium fluoride (IV) does protect against acid attack, as the PF-factor suggests.

Measurement of Hardness. Experiment 2: Phosphopeptides with Calcium Chelating Components & Amino-Peptides.
Code: Product number (A), Experimental product (B), delta P μm (C), PF factor (D), % peptide/water w/w (E), % carbodiimide/water (F),% CaCl₂.2H₂O/water (G), % KH₂PO₄/water (H), pH (hardness test)(I).

| A | B | C | D | E | F | G | H | I |
|---|---|---|---|---|---|---|---|---|
| 1 | Casein phosphopeptide (CPP) | 43** | 20.4 | 6.8 | | 26.3 | 14.6 | 6.8 |
| 2 | Polymerized (CPP) = (CPP)ₙ | 22.6* | 58.1 | 7.2 | 7.6 | 27.9 | 15.2 | 6.9 |
| | | 37 | 31.5 | 7.4 | 2 | 28.8 | 15.7 | 8.1 |
| 3 | Natural phosvitin (Phos) | 22.3* | 58.7 | 7.1 | — | 25.4 | 14.1 | 7.2 |
| 4 | Hydrolysed (Phos) = (PPP) | 51 | 5.6 | 6.3 | — | 24.1 | 13.4 | 7.2 |
| 5 | Partially hydrolyzed phosvitin, (Phos- | 8.2 | 84.8 | 7.2 | — | 7.2 | 3.6 | 7.2 |
| | h) | 7.2 | 86.7 | 7.5 | — | 7.2 | 3.6 | 7 |
| 6 | Epsilon-polylysine | 23.1 | 57.2 | 2.3 | — | 0 | 0 | 7.3 |
| | (elys; e-polylysine) | 17.9* | 66.9 | 3.6 | — | 0 | 0 | 9 |
| | | 14.4 | 73.3 | 3.6 | — | 5.6 | 3.1 | 9 |

Remark: All dosages are made on weight basis.
**: average of three measurements; *: average of two measurements.
“% carbodiimide/water”: the amount of 1-ethyl-3-(3-dimethylaminopropyl-carbodiimide that has been used in the polymerization reaction.
Product 4 (PPP): phosvitin has been partially dephosphorylated and subsequently treated with trypsin; Mw 1-3 kD.
Product 5 (Phos-h): phosvitin has been partially hydrolyzed with trypsin and ultrafiltrated on a membrane with cut off 10 kD; (Phos-h) can contain component gln49-arg212 (Mw>20 kD) of phosvitin (ref. A. Goulas, journal of Protein Chemistry, vol 15, no. 1, 1-9, 1996).

It is known in the prior art that CPP has anticariogenic competence and that it has the ability to accumulate on the tooth surface. However it appears that polymerized CPP, so-called (CPP)ₙ, has a higher PF-factor and is a superior protector. Phosvitin (Mw>30 kD) is larger compared to CPP (Mw 1-2 kD) and has a PF-factor comparable to the one of polymerized CPP. The hydrolyses of phosvitin to a low molecular weight peptide (PPP, 1-3 kD) effects the PF value drastically. The effect of size on the PF value is demonstrated both with CPP and phosvitin (product 1,2,3,4).

The removal of a small piece from phosvitin, with retention of large pieces (e.g. gln49-arg212), provides a product with the best PF factor within the class of phosphopeptides (product 5, PF:85); it is superior to the protection competence of natural phosvitin. The PF value of epsilon-polylysine (6) is twice as high as the one of Tris(hydroxymethyl)aminoethane (V), at similar molar concentration of the amino-function. It demonstrates once more the importance of using products with higher molecular weight.

Measurement of Hardness. Experiment 3:
Aminopeptides & Saccharides with Calcium Chelating Components
Code: Product number (A), Experimental product (B), delta P μm (C), PF factor (D), % (peptide+saccharide)/water w/w (E), ratio peptide/amino-polymer (F), % carbodiimide/water (G),% CaCl₂.2H₂O/water (H), % KH₂PO_{4/}water (I), pH (hardness test)(J).

| A | B |
|---|---|
| 7 | Casein phosphopeptide × e-polylysine copolymer (CPP × elys)ₙ |
| 8 | Casein phophopeptide × e-polylysine copolymer (CPP × elys)ₙ |
| 9 | Casein phosphopeptide × e-polylysine copolymer (CPP × elys)ₙ |
| 10 | (CPP × elys)ₙ purification with ultrafiltration |
| 11 | (CPP × elys)ₙ + trizma base |
| 12 | (CPP × elys)ₙ + 0.1% sodium fluoride |
| 13 | Casein phosphopeptide × hydrolysed-chitosan (CPP × hy-chit)ₙ |
| 14 | Casein phosphopeptide × hydrolysed-chitosan (CPP × hy-chit)ₙ |
| 15 | Casein phosphopeptide/hydrolysed chitosan mixture |
| 16 | Phosvitin hydrolysate × e-polylysine copol. (Phos-h × elys)ₙ |

| A | C | D | E | F | G | H | I | J |
|---|---|---|---|---|---|---|---|---|
| 7 | 11.9** | 77.9 | 3.6 | 10/4.1 | 2.8 | 5.6 | 3.1 | 7.1 |
| 8 | 3.1 | 94.3 | 7.8 | 10/4.1 | 5.9 | 12.2 | 6.7 | 7.2 |
| 9 | 0.65* | 98.8 | 8.1 | 10/4.1 | 6.2 | 12.8 | 7 | 7.7 |
| 10 | 2 | 96.3 | 7.8 | 10/4.1 | 1.25 | 6.1 | 3.4 | 8.5-9 |
| 11 | 3 | 94.4 | 7.8 | 10/4.1 | 1.25 | 6.1 | 3.4 | 8.5-9 |
| 12 | 0 | 100 | 3.6 | 10/4.1 | 2.8 | 5.7 | 3.1 | 7.5 |
| 13 | 20* | 63 | 2.2 | 10/2.3 | 2.4 | 7.1 | 3.9 | 7.3 |
| 14 | 2.5* | 95.5 | 8.4 | 10/2.3 | 8.9 | 26.7 | 13.3 | 7 |
| 15 | 30.5 | 43.5 | 2.3 | 10/2.3 | 2.4 | 7.2 | 4 | 7.4 |
| 16 | 15.9 | 70.6 | 3.6 | 10/4.1 | — | 5.7 | 3.1 | 7.2 |

Remark: (**): average of three measurements; (*): average of two measurements. Copolymerization of casein phosphopeptide with epsilon-polylysine provides products with higher PF-value in comparison to polymerized casein phosphopeptide (product 7,8,9 versus 2). The larger scale production, product (10), which has been ultrafiltrated, delivers a product that is purified from small products, originating from the hydrolyses of the carbodiimide. The comparison of the PF-value of (10) with the one of products (7), (8) and (9) demonstrates the limited influence of the small products on the PF-value. Also the addition to product (10) of a small sized base, such as Trizma, does not provide improved protection competence. The effect of (CPP x elys)ₙ and sodium fluoride is additive (product 7 and 12).

The use of such mixture allows to completely neutralize the effect of 0.1 N acetic acid on the hardness of the tooth surface at a low peptide dosage level (3.6%). In principle it is possible to replace epsilon-polylysine with water soluble hydrolyzed chitosan (Mw<30000 dalton) (product 13 and 14). Product 13, the casein phosphopeptide hydrolyzed chitosan copolymer, contains the same ingredients as product (15); but they differ in the fact that the carbodiimide (of 15) has been hydrolyzed in water before the phosphopeptide and chitosan have been added, resulting in absence of coupling activity. The difference in PF-value between (13) and (15) demonstrates the importance of coupling.

Code: Productnumber (A), Experimental product (B), delta P μm (C), PF factor (D), % peptide+saccharide/water w/w (E), number of reaction stages (F),% CaCl₂.2H₂O/water (G), % KH₂PO_{4/}water (H), pH (hardness test)(I).

| A | B |
|---|---|
| 17 | bisphosphonylated e-polylysine (low peroxide dosage) |
| 18 | bisphosphonylated e-polylysine (high peroxide dosage) |
| 19 | bisphosphonylated e-polylysine (coupling at basic pH) |
| 20 | bisphosphonylated e-polylysine (coupling at acid pH) |
| 21 | bisphosphonylated e-polylysine (coupling at acid pH) |
| 22 | bisphosphonylated e-polylysine (large scale, ultrafiltration) |
| 23 | bisphosphonylated e-polylysine (coupling in water) |
| 24 | denaturated bisphosphonylated e-polylysine (coupling acid pH) |
| 25 | denaturated bisphosphonylated e-polylysine (coupling acid pH) |
| 26 | carboxylated chitosan |
| 27 | 3-hydroxyphthalated e-polylysine |

| A | C | D | E | F | G | H | I |
|---|---|---|---|---|---|---|---|
| 17 | 9.8 | 81.9 | 3.5 | 1 | 5.7 | 4.6 | 8.8 |
| 18 | 7* | 87 | 3.5 | 1 | 5.7 | 3.2 | 8.8 |
| 19 | 2.5 | 95.4 | 3.5 | 2 | 5.7 | 3.2 | 9.4 |
| 20 | 0 | 100 | 3.5 | 2 | 5.7 | 3.2 | 9 |
| 21 | 2 | 96.3 | 3.1 | 2 | 5 | 2.7 | 8.9 |
| 22 | 2 | 96.3 | 3.6 | 2 | 2.8 | 1.5 | 9 |
| 22 | 16 | 70.4 | 1.5 | 2 | 2.8 | 1.5 | 9 |
| 23 | 3.8 | 93.1 | 3.5 | 2 | 5.7 | 3.2 | 9.1 |
| 24 | 6 | 88.9 | 1.5 | 2 | 1.2 | 0.7 | 9.4 |
| 25 | 0.6 | 99 | 1.5 | 2 | 1.2 | 0.7 | 9.2 |
| 26 | 52.8 | 2.8 | 3.1 | | 1.6 | — | 7.7 |
| 27 | 5 | 90.7 | 3.6 | | 5.6 | 3.1 | 8.7 |

Remark: (**): average of two measurements.

Bisphosphonylated epsilon-polylysine, products 17 to 21 and 23, have been purified with GPC (Sephadex G-25); the products 22, 24 and 25 have been ultrafiltrated (Millipore prepscale cellululose cartridge cut-off 1000 dalton). The addition of bisphosphonate groups on epsilon-polylysine increases the PF-value, both in 1-step and 2-step procedures (product 6,17 to 25). The 1-step procedure is carried out with excess of hydrogen peroxide. The 2-step procedure provides products with higher PF-value compared to products made with the 1-step procedure. It is advised to remove the excess of hydrogen peroxide before the epoxide is allowed to react with epsilon-polylysine (product 17 and 18 versus 19 to 22). Bisphosphonylated epsilon-polylysine exhibits high PF-values, irrespective of the purification procedure (gel permeation chromatography or ultrafiltration; product 22). The addition of the epoxide to epsilon-polylysine in water or water/alcohol mixtures provides products with high PF-value (product 19 to 23). The use of denaturated epsilon-polylysine instead of non-denaturated epsilon-polylysine in the reaction with the expoxide, provides products with higher-PF value (product 25, 24 and 22). The addition of 3-hydroxyphthalate groups to epsilon-polylysine is increasing the PP-value (product 6 and 27).

Experiment 4: Gelled Formulations that Contain Aminopeptides (with Calcium Chelating Components) and Fluoride.

Tooth samples have been treated (the procedure is outlined under chapter “Examples; Method & Materials; determination of the PF-value”) with gels that have been viscosfied with hydroxyethylcellulose. They contained sodium fluoride, CaCl₂.2H₂O and KH₂PO₄, and optionally bisphosphonylated e-polylysine and/or casein phosphopeptide-epsilon-polylysine copolymer.

| | bisphosphonated | CPP e-polylysine | | | | | | |
|---|---|---|---|---|---|---|---|---|
| cellulose | e-polylysine | copolymer | NaF | CaCl2•2H20 | KH2PO4 | delta P | | |
| % | % | % | % | % | % | μm | PF | pH |
| 4.0 | 0 | 0 | 0 | 0 | 0 | 55 | 0 | 9.0 |
| 4.2 | 0 | 0 | 0.3 | 0 | 0 | 18.8 | 68.1 | 8.8 |
| 4.2 | 4.2 | 0 | 0.3 | 3.3 | 1.8 | 10.7 | 81.9 | 9 |
| 0 | 4.0 | 7.0 | 0.3 | 3.2 | 1.7 | 6 | 89.9 | 7.7 |
| 4.0 | 7.0 | 0 | 0.3 | 5.5 | 3.0 | 4 | 93.2 | 9.1 |

%: on weight basis versus water
cellulose: hydroxyethylcellulose; all solutions are gelled.
CPP-e-polylysine copolymer/Ca/PO4 mixtures are viscous as such and do not necessarily require a thickener to prepare a gel; Bisphosphonylated e-polylysine/Ca/PO4 mixtures are not viscous
pH: of the gelled solution

Three tooth samples have been treated with each of the gels (except for the gel that contains hydroxyethylcellulose only); data on hardness are average values.

### C.2. In-Vivo Results

### Production of Tooth Samples for use in In-Vivo Tests:

Tooth samples have been cut in horizontal slices (thickness: 0.3 to 1 mm) with a Leitz 1600 tooth cutter with horizontal cutting blade; the slices are cut manually (with a bore) into small tooth samples that contain a small part of the original tooth surface. The tooth sample is positioned in a small plastic tube, that has been cut from a long plastic tube with 6 mm external diameter. The length of the tube is 3 to 6 mm. The inner part of the tube is filled with a polymer that hardens under light (Photoclearfil Bright; Kuraray). The tooth sample is positioned in the soft polymer inside the tube, in such a way that the original tooth surface is just surfacing above the tube. Finally the polymer is hardened with a light source. The tooth surface is polished with Struers silicon carbid paper (800-4000) and the hardness is measured 5 times on each of the samples under the Letiz Wetzlar microscope (weight on needle: 50 p). Tooth samples with a maximum average depth of penetration of 43 μm are retained for use in the oral cavity.

The tooth-samples are fixed to a device that has been described in the chapter “Description of the invention ( chapter: In-vivo experiments)” and the device is positioned in the oral cavity after the lower jaw.

C.2.1. Protection of Radiated Tooth-Samples that Reside 5Days in the Oral Cavity with Bisphosphonylated E-Polylysine or with Fluoride.

Tooth samples have been subjected to a radiation dose that is equal to the amount that is given to patients with oral cancer (20 Gray) and have been sterilized. Subsequently, the samples have been treated either with a solution of 0.1% NaF or with an aqueous solution of 4.0% bisphosphonylated epsilon-polylysine, 3.1% CaCl₂.2H₂O and 1.7% KH₂PO_{4.} A drop of the solution was positioned on the tooth surface and allowed to reside for a period of 30 minutes at 37 C.; the procedure was repeated after removal of the drop with pressured air. Finally the drop was removed, the tooth was dried on air and fixed to the device. In total six tooth samples have been fixed to the device and positioned in the oral cavity after the lower jaw. It remained in the oral cavity for a period of 5 days (day and night). After completion of the required residence time, the device was removed from the oral cavity and the experimental teeth where separated from the device and washed. The hardness was measured according to the Knoop method, six times on each of the tooth samples.

| A | B | N | n | Min | Max | Mean | St. Dev. |
|---|---|---|---|---|---|---|---|
| blanco | start | 24 | 144 | 39.90 | 43.00 | 40.80 | 0.862 |
| | after radiation | 24 | 144 | 39.60 | 42.90 | 40.89 | 0.911 |
| | after 5 days oral cavity | 24 | 144 | 41.30 | 49.30 | 44.54 | 1.954 |
| 0.1% NaF | start | 23 | 138 | 39.70 | 42.50 | 41.10 | 0.924 |
| | after radiation | 24 | 144 | 39.30 | 43.00 | 40.97 | 0.950 |
| | after 5 days oral cavity | 24 | 144 | 40.00 | 45.60 | 42.77 | 1.516 |
| 4% bisphosphonate- | start | 24 | 144 | 39.80 | 42.40 | 40.87 | 0.739 |
| e-polylysine | after radiation | 24 | 144 | 39.80 | 44.00 | 41.02 | 1.031 |
| | after 5 days oral cavity | 24 | 144 | 39.10 | 48.20 | 41.97 | 1.858 |

A: the type of protector that has been used; the formulation with 4% bisphosphonylated epsilon-polylysine, did contain also CaCl₂ and KH₂PO₄.
B: the stage at which the hardness of the tooth surface has been measured with the Knoop method.
(before the treatment; after radiation and before addition of protector formulation to the surface; after addition of protector to the surface of the tooth and after residing five days in the oral cavity).
N: the number of tooth samples in the oral cavity (six per person; 4 volunteers; 24 samples per type of solution; 72 tooth samples have been used and randomized into twelve groups with the same average hardness value).
n: the number of hardness measurements per group of tooth samples (six per tooth)
Min: minimum hardness found in the group (μm)
Max: maximum hardness found in the group (μm)
Mean: Average hardness found in the group (μm)
St.Dev.: standard deviation

| B | A | N | delta |
|---|---|---|---|
| after radiation | blanco | 24 | 0.09 |
| | 0.1% fluoride | 23 | −0.13 |
| | 4% bisphosphonate e-polylysine | 24 | 0.15 |
| radiation+ | blanco | 24 | 3.74 |
| 5 days in oral cavity | 0.1% fluoride | 23 | 1.67 |
| | 4% bisphosphonate e-polylysine | 24 | 1.10 |

B: the stage at which the hardness of the tooth surface has been measured with the Knoop method.
(after radiation and before addition of the formulation to the surface of the tooth; after addition of the formulation to the surface and after residing five days in the oral cavity)
A: the type of protector that has been used; the formulation with 4% bisphosphonylated epsilon-polylysine, contained also CaCl₂ and KH₂PO₄.
N: the number of tooth samples in the oral cavity (six per person)
delta: change of hardness (μm) due to radiation or due to radiation+5 days residence in the oral cavity.

Statistical test: Mann-Whithey - p-value

| | 0.1% fluoride | 4% bisphosphonate e-polylysine |
|---|---|---|
| blanco | 0.001 | 0.000 |
| fluoride | — | 0.036 |

Some of the radiated tooth samples that where recovered after 5 days in the oral cavity have been subjected to an additional treatment with 1 drop of acetic acid (0.1N; pH 5) for a period of 30 min. at 37° C.; the acid was washed away, the tooth was dried and the hardness was measured.

| type of protector | N | A | B | C |
|---|---|---|---|---|
| blanco | 5 | 43.3 | 53.4 | 10.1 |
| 0.1% fluoride | 3 | 41.9 | 48.4 | 6.5 |
| 4% bisphosphonate e-polylysine | 5 | 40.6 | 44.5 | 3.9 |

N: number of tooth samples
A: hardness(μm) after radiation and 5 days residence in the oral cavity
B: hardness after radiation and 5 days in the oral cavity and treatment with acetic acid

### C: B−A

Statistical test: Mann-Whithey—p-value

| | 0.1% fluoride | 4% bisphosphonate e-polylysine |
|---|---|---|
| blanco | 0.25 | 0.032 |

C.2.2. Protection of Tooth Samples that Reside 12 Hours in the Oral Cavity.

A drop from a solution containing either, e-polylysine, CPP, bisphoshphonylated e-polylysine, casein phosphopeptide epsilon-polylysine copolymer or partially hydrolysed phosvitin has been positioned at the surface of tooth samples for a period of 30 minutes at 37 C.; the drop was removed and the procedure was repeated once more. All solutions contained also 3.1% CaCl₂.2H₂O and 1.7% KH₂PO4 (except for the one containing e-polylysine).

The tooth surface was cleaned with a soft paper towel until it was visibly clean. Four such tooth samples where fixed to the device and located in the oral cavity behind the lower jaw. They resided in the oral cavity for a period of 12 hours (only to be removed during eating). Subsequently, they where removed, cleaned and the tooth surface was treated with a drop of acetic acid (0.1N, 30 min., 37° C.); the acid was removed, the tooth was washed with deionized water and the hardness was evaluated.

| A | B | C | D | E | F |
|---|---|---|---|---|---|
| blanco (saliva) | 40.63 (1.05) | 49.45 (2.23) | 8.82 | — | 16 |
| 4.2% e-polylysine | 40.88 (0.91) | 47.81 (3.67) | 6.93 | 0.200 | 20 |
| 4.2% casein phosphopeptide | 40.82 (0.73) | 47.60 (2.53) | 6.78 | 0.073 | 16 |
| 4.2% bisphosphonated e-polylysine | 40.52 (0.62) | 44.29 (2.08) | 3.77 | 0.000 | 13 |
| 4.2% bisphosphonated e-polylysine (*) | 40.76 (0.73) | 44.05 (2.92) | 3.29 | 0.000 | 16 |
| 4.2% CPP-e-polylysine copolymer | 40.81 (0.68) | 46.21 (1.94) | 5.40 | 0.000 | 16 |
| 7.8% CPP-e-polylysine copolymer | 40.51 (1.55) | 42.92 (1.43) | 2.41 | 0.000 | 13 |
| 7.5% partially hydrolysated phosvitin | 40.76 (0.80) | 46.08 (3.32) | 5.32 | 0.004 | 16 |

A: type of peptide ingredient in the solution (% on a w/w basis versus water).
(*): the teeth where submerged (30 min.) in a saliva batch and dried afterwards on air, before they where treated with a solution that contained bisphosphonylated e-polylysine.
B: Average initial hardness of the tooth surface; μm (st. dev.)
C: Average hardness after treatment, after residing in the oral cavity and treatment with acid
D: The average change in hardness (C−B)
E: p-value (against blanco) of the Mann-Whitney statistical test
F: the number of tooth samples that where treated with a specific formulation; hardness on each of the tooth samples was measured five times and an average value was calculated; most often 4 tooth samples per person; 5 volunteers participated to the experiment; the 132 tooth samples where randomized into groups (of 4 teeth) with the same average hardness value.

1. Peptides that are able to form a complex with calcium, that contain phosphonate—or phosphate groups, and that are capable to protect teeth against acid attack, chosen from the group consisting of:
bisphosphonylated-epsilon-polylysine (Bispho x elys),
casein phosphopeptide epsilon-polylysine copolymer (CPP x elys)ₙ,
hydrolyzed phosvitin that has been conjugated to epsilon polylysine (Phos-h x elys)ₙ,
casein phosphopeptide that has been polymerized with a carbodiimide (CPP)ₙ and
phosvitin that has been hydrolyzed with trypsin, pepsin or a combination of both (Phos-h).

2. Chitosan hydrolysate, with a molecular weight of maximum 30 kD, that has been conjugated with casein phosphopeptide (CPP) into (CPP x hv-chit)ₙ.

3. The use of amino-proteins that contain one or more components able to complex calcium ions, phospho-proteins that contain one or more components able to complex calcium ions, hydrolyzed chitosan that contains one or components able to complex calcium ions and/or amino-proteins or a mixture of such products to protect teeth against acid attack and/or to control the bacterial flora in the oral cavity,
wherein:
a) the amino-proteins that contain one or more components able to complex calcium ions are chosen from the group consisting of, bisphosphonylated epsilon-polylysine (Bispho x elys), biscarboxylated epsilon-polylysine, 3-hydroxy-phthalated epsilon-polylysine, proteins that are bisphosphonylated or biscarboxylated, and that have a molecular weight of at least 2 kD and contain at least 40% of the amino acid lysine,
b) the phospho-proteins that contain one or more components able to complex calcium, are chosen from the group consisting of, polymerized casein phosphopeptide (CPP)ₙ, partially hydrolyzed phosvitin (Phos-h), casein phosphopeptide-epsilon-polylysine-copolymer (CPP x elys)ₙ, and copolymers of hydrolyzed phosvitin (Phos-h) or phosvitin (Phos) with epsilon-polysine or with hydrolyzed chitosan to respectively (Phos-h x elys)ₙ, (Phos-h x hy-chit)ₙ, (Phos x elys)ₙ and (Phos x hy-chit)ₙ,
c) the hydrolyzed chitosan that contains one or more components able to complex calcium is, bisphosphonylated-hydrolyzed chitosan (Bispho x hy-chit) or casein phosphopeptide-hydrolyzed-chitosan-copolymer (CPP x hy-chit)ₙ, and
d) the amino-proteins are, epsilon-polylysine (elys) or polylysine.

4. The use according to claim 3. wherein said proteins are peptides.

5. The use of epsilon-polylysine or of polylysine according to claim 3. for the protection of teeth, for the control of the bacterial flora in the oral cavity or to treat halitosis.

6. The use of a phosvitin hydrolysate according to claim 3. wherein said phosvitin hydrolysate is obtainable by treating phosvitin with one or more proteases.

7. The use according to claim 6 wherein said phosvitin hydrolysate is obtainable by hydrolysing phosvitin with trypsin, chymotrypsin, pepsin, or a combination of said enzymes.

8. The use according to claim 3 wherein said bisphosphonylated epsilon-polylysine is 2-e-polylysine-1-hydroxyethane-1,1-diphosphonate , whereby the number of bisphosphonyl groups varies between one and the amount of amino groups that is present in the peptide.

9. The use according to claim 3 wherein said hydrolyzed chitosan has a molecular weight that is lower or equal to 30 kD, and is obtainable by hydrolyzing chitosan by an acid or an enzyme.

10. A method to produce polymerized casein phosphopeptide (CPP)ₙ in water characterized in that: casein phosphopeptide is polymerized with a water-soluble carbodiimide.

11. The method according to claim 10 wherein said carbodiimide is 1-ethyl-3-(3-dimethylaminopropyl)-carbodiimide.

12. A method to produce casein phosphopeptide x epsilon-polylysine-copolymer (CPP x elys)ₙ, in water characterized in that: casein phosphopeptide and epsilon-polylysine are conjugated with a water soluble carbodiimide.

13. The method according to claim 12 wherein said carbodiimide is 1-ethyl-3-(3-dimethylaminopropyl)-carbodiimide.

14. The method to produce casein phosphopeptide x hydrolyzed-chitosan-copolymer (CPP x hy-chit)ₙ in water characterized in that: casein phosphopeptide and hydrolyzed chitosan (with a molecular weight below or equal than 30 kD) are conjugated with a water soluble carbodiimide.

15. The method according to claim 14 wherein said carbodiimide is 1-ethyl-3-(3-dimethylaminopropyl)-carbodiimide.

16. The method to produce a copolymer from hydrolyzed phosvitin and epsilon-polylysine (Phos-h x elys)ₙ characterized in that: phosvitin has been hydrolyzed with pepsin, trypsin, chymotrypsin (or a combination of such enzymes), and has been conjugated to epsilon-polylysine with a water-soluble carbodiimide such as 1-ethyl-3-(3-dimethylaminopropyl)-carbodiimide and/or with glutaminase.

17. The method to produce bisphosphonylated epsilon-polylysine from a mixture of hydrogen peroxide, epsilon-polylysine and vinylidene diphosphonate (in salt or acid form).

18. The method to produce bisphosphonylated epsilon-polylysine characterized in that: epsilon-polylysine is allowed to react with a bisphosphonylated epoxide at a pH from 3 to 9.

19. The method according to claim 18 wherein some of the substituents on the epoxide consists of hydrogen or alkyl groups and wherein the phosphonyl groups are esterified or exist in the acid (H⁺) or salt (Na⁺, K⁺, other) form or a mixture.

20. The method according to claim 18 wherein the bisphosphonylated epoxide is epoxyethane-1,1-diphosphonate.

21. The method according to claim 18 wherein the reaction is carried out at a pH of between 3 to 6 and with a BF₃ catalyst.

22. The method according to claim 18 wherein the reaction is carried out in water or in a mixture of water and alcohol.

23. The method according to claim 22 wherein the term alcohol refers to methanol, ethanol, isopropanol or butanol.

24. The method to produce bisphosphonylated epsilon-polylysine characterized in that: epsilon polylysine, that has been denaturated with a denaturating agent such as urea, is allowed to react with a bisposphonylated epoxide at a pH of between 3 to 9.

25. The method to produce 3-hydroxy-phthalated epsilon-polylysine from epsilon-polylysine and 3-hydroxyphthalic anhydride.

26. The use of the compounds indicated in claims 1 to 9 as ingredient or combination of ingredients, in products for oral care such as toothpaste, mouth-refreshing solution, mouth sprays and gets, chewing gum, candies and other food systems, artificial saliva's, medical oral care products for the treatment of teeth from patients with xerostomia, oral cancer, Hodgkin's disease, Sjögren syndrome, HIV, diabetes.

27. The use of the compounds indicated in claims 1 to 9, according to claim 26, in combination with additional ingredients such as fluoride, anticariogenic sugars, peptides for remineralisation, antibacterial products, vaccins, anti-bodies, acid absorbing ingredients, encapsulated ingredients, thickeners, anionic, nonionic, cationic or amphoteric detergents, humidifiers, abrasive ingredients, anti-tooth stone, aroma's, preservatives, cooling agents, anti-sensitive ingredients and/or sweeteners.
